(19) United States
(12) Patent Application Publication
(10) Pub. No.: US 2025/0134902 A1
(43) Pub. Date: May 1, 2025

(54) DRUG REPURPOSING FOR DELAYED TREATMENT OF ISCHEMIC STROKE

(71) Applicant: UNIVESRITY OF NORTH TEXAS HEALTH SCIENCE CENTER, Ft. Worth, TX (US)

(72) Inventors: Derek SCHREIHOFER, Ft. Worth, TX (US); John Schetz, Ft. Worth, TX (US)

(21) Appl. No.: 18/726,054

(22) PCT Filed: Feb. 9, 2023
(86) PCT No.: PCT/US2023/062282

§ 371 Date: Jul. 1, 2024

Related U.S. Application Data
(60) Provisional application No. 63/308,259, filed on Feb. 9, 2022.

(51) Int. Cl. A61K 31/5415; A61K 31/135; A61K 31/215; A61K 45/06; A61P 9/10

(52) U.S. Cl. CPC A61K 31/5415; A61K 31/135; A61K 31/215; A61K 45/06; A61P 9/10

(57) ABSTRACT

A method for treating ischemic injury in a subject having an ischemic injury is disclosed. The method can include administering to the subject a composition comprising an effective amount of a Sigma receptor agonist to treat the ischemic injury in the subject. The composition can be administered at least about 6 hours after occurrence of the ischemic injury, and administration of the composition can stimulate release of brain-derived neurotropic factor from neurons and/or to reduce one or more neurological deficits and/or symptoms.

### CROSS REFERENCE TO RELATED APPLICATIONS

This application claims the benefit of priority of U.S. Provisional Patent Application No. 63/308,259 filed Feb. 9, 2022. The contents of the referenced application are incorporated into the present application by reference.

### STATEMENT REGARDING FEDERALLY SPONSORED RESEARCH

This invention was made with government support under NS095271 awarded by the National Institutes of Health. The government has certain rights in the invention.

### BACKGROUND OF THE INVENTION

### A. Field of the Invention

The present disclosure generally relates to Sigma receptor agonists and uses thereof to treat ischemic injury in a subject. In one aspect, the Sigma receptor agonists can include Sigma-1 receptor (S1R) agonists. The Sigma receptor agonists can be administered during the chronic phase of the ischemic injury.

### B. Description of Related Art

Cerebral ischemia, also known as brain ischemia or cerebrovascular ischemia, occurs when there is an insufficient amount of blood flow to the brain. Oxygen and vital nutrients are carried in the blood through arteries, and the arteries that provide blood to the brain follow a certain pathway that ensures the brain is adequately supplied with blood. When an artery in the brain becomes blocked or bleeds, this leads to cerebral hypoxia, or a lower oxygen supply, to the region of the brain that relies on that particular artery. Even a temporary deficit in oxygen supply can impair the function of the oxygen-deprived region of the brain, and if brain cells are deprived of oxygen for more than a few minutes, severe damage can occur, which may result in the death of the brain tissue, a cerebral infarction, or ischemic stroke.

There are various drugs currently available to treat ischemic injury. However, these drugs are used shortly after occurrence of the ischemic injury event. By way of example, Alteplase is an anti-thrombolytic drug that can be used to dissolve blood clots to help reduce ischemic injury. In order for Alteplase to be effective, it is administered as soon as possible but within 3 hours after symptom onset (i.e., during the hyper-acute phase of ischemic injury). There are other drugs for treating acute ischemic injury. These drugs have been successful in pre-clinical studies but have failed in clinical trials. This suggests that acute ischemic injury may not be a beneficial target for pharmacological intervention. Additionally, administration within this narrow range of time presents problems from a timing perspective—e.g., patients may not realize they are having an ischemic injury event in the time period needed to administer the drug.

In response, the medical community has focused their efforts on the use of non-pharmacological/non-drug-related treatments to ameliorate ischemic injury symptoms that persist during the later phases of ischemic injury. Examples include movement therapy, speech therapy, mental practice therapy, and combinations of such therapies. These treatment methods can be costly and time-consuming, which can result in reduced patient compliance with the aforementioned treatment methods.

### SUMMARY OF THE INVENTION

A discovery has been made that provides a solution to at least one or more of the problems associated with treating ischemic injuries (e.g., ischemic stroke). In one aspect, a solution resides in the use of therapeutics capable of being repurposed for treatment and recovery beginning during the acute phase and continuing into the chronic phase of ischemic injury. Drugs with existing regulatory approval can be screened for activity at potential targets for treating ischemic injury. Therapeutic targets can include, for example, Sigma receptors, preferably Sigma-1 receptors (SIR), which can modulate the actions of neurotransmitter receptors, ion channels, and synaptic function, and are involved in the regulation of diverse processes such as neuroprotection, neurorestoration, neuroplasticity, and neurotransmitter release. The present disclosure is the first demonstration that oral dosing with an approved S1R agonist in a preclinical ischemia model can effectively improve neurological outcomes.

Without wishing to be bound by theory, it is believed that the therapeutic effect of S1R ligands likely results from the pleiotropic nature of the S1R, including not only modulation of ion channels and neurotransmitter receptors, but also intracellular calcium and endoplasmic reticulum (ER) stress, reductions in nitrosative stress, increases in brain-derived neurotropic factor (BDNF) and its receptor TrkB, and increases in antiapoptotic proteins such as Bcl2. Regulation of BDNF is particularly important as it is involved in the maintenance and repair of neurons, synaptic plasticity, and learning and memory. For example, as illustrated in a non-limiting manner in the Examples, S1R agonists stimulated release of BDNF from neurons in vitro and dose-dependently improved global neurological deficits in vivo over 12 days. Accordingly, in some aspects, the present disclosure supports the role of S1R agonists with the ability to improve stroke outcomes when administered during the acute phase and continuing into the chronic phase of stroke.

The use of Sigma receptor agonists can therefore provide an efficacious treatment to support recovery beginning during the acute phase and continuing into the chronic phase of ischemic injuries, including ischemic stroke, at least by reducing neurological deficits or symptoms. This can be advantageous, as it reduces the risk of patient non-compliance with known ischemic injury treatment methods. Additionally, it can reduce the need of quickly administering drugs during the hyper-acute phase of ischemic injury.

In some aspects of the invention, methods of treating ischemic injury in a subject having an ischemic injury are described. A method can include administering to the subject a composition comprising an effective amount of a Sigma receptor agonist to treat the ischemic injury in the subject. In some embodiments, the composition is administered at least about 6 hours after occurrence of the ischemic injury. In some embodiments, the composition comprises an effective amount of the Sigma receptor agonist to stimulate release of brain-derived neurotropic factor from neurons and/or to reduce one or more neurological deficits and/or symptoms.

In some embodiments, the ischemic injury is induced by a cerebral ischemia. In some embodiments, the cerebral ischemia is thrombotic ischemia, embolic ischemia, or hypoperfusion. In some embodiments, the cerebral ischemia is ischemic stroke. In some embodiments, the ischemic injury is an acute ischemic injury. In some embodiments, the ischemic injury is a chronic ischemic injury.

In some embodiments, the Sigma receptor agonist is a Sigma-1 receptor agonist. In some embodiments, the Sigma receptor agonist comprises an antitussive, an antipsychotic, an antidepressant, a neurosteroid, a neuroleptic, or a psychostimulant, or any combination thereof. In specific embodiments, the Sigma receptor agonist comprises oxeladin. Additionally or alternatively, in specific embodiments, the Sigma receptor agonist comprises promethazine. In some embodiments, the Sigma receptor agonist crosses the subject's blood-brain barrier.

In some embodiments, the method further comprises administering the composition during a hyper-acute phase of the ischemic injury. In some embodiments, the method further comprises administering the composition during an acute phase of the ischemic injury. In some embodiments, the method further comprises administering the composition during a subacute phase of the ischemic injury. In some embodiments, the method further comprises administering the composition during a chronic phase of the ischemic injury. In some embodiments, the method further comprises administering the composition during a hyper-acute, acute, sub-acute, and/or chronic phase of the ischemic injury. In some embodiments, the composition is administered preferably 12 hours, more preferably 24 hours, still more preferably 36 hours, or even more preferably 48 hours after incidence of the ischemic injury. In some embodiments, the method further comprises administering the composition within 6 hours after the occurrence of the ischemic injury. In some embodiments, the method further comprises administering the composition within 48 hours after the occurrence of the ischemic injury. In some embodiments, the composition is administered one or more times daily. In some embodiments, the composition is administered at least until one or more symptoms of the ischemic injury are resolved. In some embodiments, the composition comprises a dose of 10-200 mg/kg of the Sigma receptor agonist. In some embodiments, the composition is administered orally, subcutaneously, intraperitoneally, or intravenously.

In another aspect of the present disclosure, ischemic injury-reducing compositions are described. An ischemic injury-reducing composition can comprise an effective amount of one or more Sigma receptor agonists and one or more additional therapeutic agents and/or one or more pharmaceutically acceptable excipients to treat or reduce ischemic injury. The one or more additional therapeutic agents can comprise ketamine and/or selective serotonin reuptake inhibitors. Such a composition can be obtained by methods of the present disclosure, including at least by providing an effective amount of a Sigma receptor agonist capable of treating the ischemic injury; and combining the Sigma receptor agonist with one or more additional therapeutic agents and/or one or more pharmaceutically acceptable excipients to obtain the composition.

An “acute” ischemic injury refers to the acute onset of focal neurological symptoms or deficits as a result of underlying cerebrovascular disease. Neurological symptoms or deficits can include, but are not limited to, hemiparesis, hypesthesia, hemianopsia, aphasia, speech disinhibition, dysarthria, agnosia, cortical sensory deficits, impaired or altered mental status or memory, incontinence, gait apraxia, nystagmus, diploia, vertigo, dysphagia, syncope, and/or ataxia. Acute ischemic injuries may comprise a hyper-acute phase, an acute phase, and/or a subacute phase. As used herein, the “hyper-acute phase” of an ischemic injury begins with the acute onset of focal neurological findings in a vascular territory as a result of underlying cerebrovascular disease and may end up to about 24 hours after occurrence of the ischemic injury (e.g., about 0.1 hours to about 24 hours or any range or value there between). As used herein, the “acute phase” of an ischemic injury begins about 24 hours after onset of focal neurological findings in a vascular territory as a result of underlying cerebrovascular disease and may end up to about 7 days after occurrence of the ischemic injury (e.g., about 24 hours to about 7 days or any range or value there between). As used herein, the “subacute phase” of an ischemic injury begins about 1 week after onset of focal neurological findings in a vascular territory as a result of underlying cerebrovascular disease and may end up to about 3 weeks after occurrence of the ischemic injury (e.g., about 1 week to about 3 weeks or any range or value there between).

“Chronic” ischemic injury refers to the persistence of neurological symptoms or deficits sustained after the acute phase of an ischemic injury. Neurological symptoms or deficits can include, but are not limited to, hemiparesis, hypesthesia, hemianopsia, aphasia, speech disinhibition, dysarthria, agnosia, cortical sensory deficits, impaired or altered mental status or memory, incontinence, gait apraxia, nystagmus, diploia, vertigo, dysphagia, syncope, and/or ataxia. As used herein, the “chronic phase” of an ischemic injury can begin about 3 weeks after the acute onset of focal neurological symptoms or deficits as a result of underlying cerebrovascular disease and can last weeks, months, or years after occurrence of the ischemic injury.

The terms “about” or “approximately” are defined as being close to as understood by one of ordinary skill in the art. In one non-limiting embodiment the terms are defined to be within 10%, preferably, within 5%, more preferably, within 1%, and most preferably, within 0.5%.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification, indicate an approach for preventing, inhibiting, or reducing the likelihood of the occurrence or recurrence of, a disease or condition, e.g., ischemic injury, and include any measurable decrease or complete inhibition to achieve a desired result. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

The term “subject,” as used herein, can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as ischemic injury. The subject may being undergoing or having undergone treatment. The subject may be asymptomatic. Subjects may be healthy individuals that are desirous of prevention of ischemic injury. The terms “patient” and “individual” may be used interchangeably with “subject,” in at least some cases. An individual may comprise any age of a human or non-human animal and therefore includes both adult and juveniles.

As used herein, the terms “treatment,” “treat,” or “treating” refers to intervention in an attempt to alter the natural course of the subject being treated. Treatment includes any beneficial or desirable effect on the symptoms or pathology of a disease or pathological condition, and may include even minimal reductions in one or more measurable markers of the disease or condition being treated, e.g., ischemic injury. Treatment may serve to accomplish one or more of various desired outcomes, including, for example, preventing occurrence or recurrence of disease, alleviation or reduction in severity of symptoms, and diminishment of any direct or indirect pathological consequences of the disease, preventing disease spread, delaying the rate of disease progression, amelioration or palliation of the disease state, and remission or improved prognosis. “Treatment” does not necessarily indicate complete eradication or cure of the disease or condition, or associated symptoms thereof.

The term “effective,” as that term is used in the specification and/or claims, means adequate to accomplish a desired, expected, or intended result. As used herein, the term “therapeutically effective amount” is synonymous with “effective amount,” “therapeutically effective dose,” and/or “effective dose,” and refers to an amount of an agent sufficient to produce a desired result or exert a desired influence on the particular condition being treated. In some embodiments, a therapeutically effective amount is an amount sufficient to ameliorate at least one symptom, behavior, or event, associated with a pathological, abnormal, or otherwise undesirable condition, or an amount sufficient to prevent or lessen the probability that such a condition will occur or re-occur, or an amount sufficient to delay worsening of such a condition. For instance, in some embodiments, the effective amount refers to the amount of a Sigma receptor agonist or composition thereof that can treat ischemic injury in a subject. The effective amount may vary depending on the organism or individual treated. The appropriate effective amount to be administered for a particular application of the disclosed methods can be determined by those skilled in the art, using the guidance provided herein.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other treatments for ischemic injury, can also be incorporated into the compositions.

As used herein, the terms “reference,” “standard,” or “control” describe a value relative to which a comparison is performed. For example, an agent, subject, population, sample, or value of interest is compared with a reference, standard, or control agent, subject, population, sample, or value of interest. A reference, standard, or control may be tested and/or determined substantially simultaneously and/or with the testing or determination of interest for an agent, subject, population, sample, or value of interest and/or may be determined or characterized under comparable conditions or circumstances to the agent, subject, population, sample, or value of interest under assessment.

The use of the words “a” or “an” when used in conjunction with the term “comprising,” “including,” “containing,” or “having” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The process of the present invention can “comprise,” “consist essentially of,” or “consist of” particular ingredients, components, compositions, etc., disclosed throughout the specification.

The processes and compositions of the present invention can “comprise,” “consist essentially of,” or “consist of” particular ingredients, components, compositions, etc. disclosed throughout the specification. With respect to the transitional phrase “consisting essentially of,” in one non-limiting aspect, a basic and novel characteristic of the compositions and processes of the present invention are their ability to treat or reduce ischemic injury, preferably ischemic stroke, during the chronic phase of the ischemic injury.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented below.

FIG. 1 shows competition binding of sigma receptor ligands with [³H]-(+)-pentazocine at the human Sigma-1 receptor (S1R) (FIG. 1A) and with [³H]-DTG at the human Sigma-2 receptor (S2R) (FIG. 1B).

FIG. 2 shows levels of S1R-mediated BDNF secretion from neuronal MN9D cell line as measured using in situ ELISA.

FIG. 3 shows the effects of increasing dosages of oxeladin on the food intake, water intake, and body weight male rats to determine a maximum tolerated dose of oxeladin. Rats (n=3/gp) were administered 6-150 mg/kg oxeladin citrate by oral gavage for 10 days. There was no significant effect of oxeladin on food intake (top), water intake (middle), or body weight (bottom).

FIG. 4 illustrates the pharmacokinetics in rat plasma of per oral oxeladin administration. Oxeladin (135 mg/kg) was administered by oral gavage (n=3/time point). Oxeladin was detected in the plasma and brain within 30 minutes. The half-life for both plasma and brain was greater than 4 hours.

FIG. 5 shows the effects of S1R agonists on thrombosis in rat blood. Example of the streak assay used to assess thrombotic potential of S1R ligands. Lane assignments are shown in Table 5.

FIGS. 6A-6C show the effects of oxeladin administration on neurological function after transient middle cerebral artery occlusion in rats. Oxeladin (45, 68 or 135 mg/kg/d) was administered p.o. starting on day 2 after tMCAO. FIG. 6A. The two highest doses of oxeladin significantly improved the Bederson score compared to the Vehicle control, with 135 mg/kg improving the outcomes on days 3, 7, and 14 and 68 mg/kg improving outcomes on days 7 and 14. FIG. 6B. The 135 mg/kg dose of oxeladin significantly improved the Neurological Deficit Score (NDS) on days 3 and 7 compared to the Vehicle control. FIG. 6C. The 68 mg/kg dose of oxeladin significantly improved function in the Elevated Body Swing Test (EBST) on days 7 and 14. *=P<0.05 for 135 mg/kg vs. Vehicle; †=P<0.05 for 68 mg/kg vs. Vehicle. Veh and 45 mg/kg n=10, 68 mg/kg n=11, 135 mg/kg n=12.

FIGS. 7A-7B show the effects of oxeladin administration on infarct after transient middle cerebral artery occlusion in rats. FIG. 7A. No difference in infarct size was observed by TTC staining 24 hours after a single dose of Oxeladin administered 48 hours after tMCAO (n=9/gp). FIG. 7B. Compared to Vehicle controls, Oxeladin significantly reduced (P<0.05) the loss of healthy brain tissue assessed by Nissl staining 15 days after tMCAO (n=7/gp). This reduction was evident in the infarcted hemisphere (Hem), as well as in the cerebral cortex (Cortex) and striatum (Striat).

FIGS. 8A-8G show the effects of oxeladin administration on astrogliosis after transient middle cerebral artery occlusion in rats. tMCAO significantly (t, P<0.05) increased GFAP staining in the striatum (FIGS. 8A, 8F, n=8/gp) and cortex (FIGS. 8B, 8G, n=8/gp), but not in the hippocampus (FIGS. 8C, 8E, n=4/gp) 15 days after tMCAO. Treatment with oxeladin starting 48 hours after tMCAO did not alter astrogliosis. FIG. 8D shows representative images of GFAP staining. Scale bar in FIG. 8F and FIG. 8G=50 mm.

FIGS. 9A-9G show the effects of oxeladin administration on microgliosis after transient middle cerebral artery occlusion in rats. tMCAO significantly (t, P<0.05) increased Iba1 staining in the striatum (FIGS. 9A, 9F, n=4/gp) and cortex (FIGS. 9B, 9G, n=4/gp), and hippocampus (FIGS. 9C, 9E, n=4/gp) 15 days after tMCAO. Treatment with oxeladin starting 48 hours after tMCAO did not alter astrogliosis. FIG. 9D shows representative images of Iba1 staining. Scale bar in FIG. 9F and FIG. 9G=50 mm.

FIGS. 10A-10C show the effects of oxeladin administration on BDNF levels in the brain after transient middle cerebral artery occlusion in rats. FIG. 10A. Ten days of treatment with 135 mg/kg of oxeladin p.o. (n=5) significantly increase in BDNF levels in the cerebral cortex (P<0.05) but not in the hippocampus when measured by ELISA. FIG. 10B. A single dose of 135 mg/kg oxeladin administered 48 hours after tMCAO (n=10) significantly increased (F_{1, 10}=7.685, P=0.02) the ratio of mature 17 kDa BDNF compared to 42 kDa pro-BDNF in the ischemic penumbra. FIG. 10C. Representative western blot showing proBDNF (42 kDa), mature BDNF (17 kDa) and beta actin from the ischemic penumbra of Vehicle (Veh) treated rats and rats treated with oxeladin and sacrificed 2, 6, and 24 hours after dosing.

FIGS. 11A-11G show the effects of oxeladin administration on cell proliferation in the brain after transient middle cerebral artery occlusion in rats. tMCAO significantly (†, P<0.05) increased BrdU staining in the striatum (FIGS. 11A, 11F, n=6/gp) and cortex (FIGS. 11B, 11G, n=6/gp), but not in the dentate gyrus (FIG. 11C, n=4/gp) 15 days after tMCAO. FIG. 11E shows a representative dentate gyrus with positive staining for BrdU (scale bar=50 mm). Treatment with oxeladin starting 48 hours after tMCAO did not alter BrdU staining in the striatum, but significantly reduced cortical BrdU compared to vehicle controls (FIG. 11B, **P<0.05 vs. vehicle). FIG. 11D shows representative images of BrdU staining. Scale bar in FIG. 11F=50 mm, FIG. 11E=100 mm.

FIG. 12 shows the effects of oxeladin administration on cell proliferation in the subventricular zone (SVZ) after transient middle cerebral artery occlusion in rats. Counts of BrdU positive cells in the SVZ in the infarcted hemisphere did not reveal a significant difference between vehicle (n=4) and oxeladin-treated (n=6) animals 15 days after stroke. Representative photomicrographs are shown.

### DETAILED DESCRIPTION

A discovery has been made that provides a solution to at least one or more of the problems associated with treating ischemic injuries, preferably ischemic stroke, beginning during the acute phase and continuing into the chronic phase of the injury. In one aspect, the solution can include the use of drugs with existing regulatory approval having activity at potential targets, e.g., Sigma receptors, preferably Sigma-1 receptors (SIR), for treating ischemic injury beginning during the acute phase and continuing into the chronic phase of the injury. Without wishing to be bound by theory, it is believed that the therapeutic effect of S1R ligands likely results from the pleiotropic nature of the S1R, including stimulation of BDNF release from neurons, which can support the maintenance and repair of neurons, synaptic plasticity, and learning and memory. The process of the invention is efficacious, and administration of an effective amount of a Sigma receptor agonist beginning during the acute phase and continuing into the chronic phase of ischemic injury can support recovery of a subject at least by reducing neurological deficits or symptoms to improve stroke outcomes. These and other non-limiting aspects of the present invention are described in further detail below.

### A. Ischemic Injury

The ischemic injury to be treated according to the methods of the present disclosure can be caused by diminished or absent blood flow to any tissues, muscle group, or organ of the body, and the main mechanism of injury in ischemia is hypoxia. Ischemia comprises not only insufficiency of oxygen needed for cellular metabolism, but also reduced availability of nutrients and inadequate removal of metabolic wastes. The main symptoms of ischemia include, but are not limited to, impairments in vision, body movement, and speaking, unconsciousness, blindness, problems with coordination, and/or weakness in the body. Other conditions that may result from ischemia are stroke, cardiorespiratory arrest, and irreversible brain damage.

In certain embodiments, the ischemic injury is induced by a cerebral ischemia. Cerebral ischemia or brain ischemia, is a condition that occurs when there is a lack of blood flow to the brain to meet metabolic demand. This leads to limited oxygen supply, or cerebral hypoxia, and leads to the death of brain tissue, cerebral infarction, or ischemic stroke. There are two kinds of ischemia: focal ischemia, which is confined to a specific region of the brain, and global ischemia, which encompasses wide areas of brain tissue.

With respect to cerebral ischemia, ischemic stroke can be sectioned into focal cerebral ischemia (e.g., thrombotic or embolic stroke) or global cerebral ischemia (e.g., hypoperfusion). Focal cerebral ischemia occurs when a blood clot has blocked a cerebral vessel, thereby reducing blood flow to the particular brain region and increasing the risk of cell death to that area. Thrombotic and embolic strokes are focal or multifocal in nature. Thrombotic ischemia may be caused by blockage of a blood vessel, usually due to a blood clot or a sudden spasm of an artery. Embolic ischemia may be caused by a blood clot that may have formed in the heart or an artery that then travels to another artery, causing a blockage in the destination artery. Global cerebral ischemia like hypoperfusion occurs when blood flow to the brain is stopped or reduced and can be triggered by cardiac arrest, severe blood loss from trauma, or surgery. If adequate circulation is restored within a short period of time, symptoms may be brief. However, if a large amount of time passes before restoration, brain damage can be permanent.

In some embodiments, symptoms of cerebral ischemia include, but are not limited to, weakness in one arm or leg, weakness in one entire side of the body, dizziness, vertigo, double vision, weakness on both sides of the body, difficulty speaking, slurred speech, and/or loss of coordination. The symptoms of cerebral ischemia range from mild to severe. Symptoms can last from a few seconds to a few minutes or for extended periods of time. If the brain becomes damaged irreversibly and tissue death occurs, the symptoms may be permanent.

In some embodiments, methods of the present disclosure may further comprise the step diagnosing a subject as having and/or being at risk of having an ischemic injury. Thus, in some embodiments, the subject has or is at risk of having an ischemic injury. Ischemic injury, for example, cerebral ischemia, is linked to many different diseases or irregularities, including but not limited to, for example, sickle cell anemia or other blood diseases, malformed blood vessels, arterial plaque buildup, congenital heart defects, heart disease, blood clots, irregular heartbeat, low blood pressure, heart attack, and/or ventricular tachycardia. Additional risk factors for ischemic injury, such as ischemic stroke, include, but are not limited to, high blood pressure, smoking tobacco, obesity, high cholesterol, diabetes, previous transient ischemic attack, and/or atrial fibrillation. Thus, in some embodiments, a subject at risk of having an ischemic injury may have sickle cell anemia or other blood diseases, malformed blood vessels, arterial plaque buildup, congenital heart defects, heart disease, blood clots, irregular heartbeat, low blood pressure, heart attack, ventricular tachycardia, high blood pressure, tobacco use, obesity, high cholesterol, diabetes, previous transient ischemic attack, and/or atrial fibrillation.

In order to treat cerebral ischemia, in some aspects, drugs to restore blood flow by dissolving the blood clot causing the injury (e.g., Alteplase) may be administered during the hyper-acute phase of the ischemic injury. Additionally or alternatively, there are also emergency surgical endovascular procedures in which the physician can directly treat the blocked blood vessel. In some aspects, a subject may also be prescribed additional therapeutic agents beginning during the acute phase and continuing into the chronic phase of ischemic injury. For example, systemic blood pressure should be maintained to restore blood flow to the cerebrum, and medications that can help achieve appropriate blood pressure and/or medications for lowering cholesterol or fat levels may be administered. Sometimes, after ischemic injury, subjects are at higher risk of developing seizures, and in some aspects, anti-seizure medications are administered to help prevent seizures and control seizures if they do develop.

Thus, the therapy provided herein may comprise administration of a combination of therapeutic agents, such as a Sigma receptor agonist and at least one additional therapy, e.g., blood pressure- and/or cholesterol-lowering medications, anti-seizure medications, and/or one or more additional Sigma receptor agonists (e.g., 1, 2, 3, 4, 5, or more agonists). In some embodiments, the therapy provided herein may comprise administration of a Sigma receptor agonist and one or more antitussives, antipsychotics, antidepressants, neurosteroids, neuroleptics, psychostimulants, or any combination thereof. In specific embodiments, the compositions and methods of the present embodiments involve a Sigma receptor agonist in combination with ketamine and/or selective serotonin re-uptake inhibitors.

### B. Sigma Receptors & Agonists Thereof

In some methods of the disclosure, Sigma receptor agonists of the disclosure are utilized for methods of treatment for ischemic injury in an individual in need thereof. The Sigma receptor agonists of the disclosure may or may not be utilized in therapeutic or preventative applications for a mammalian subject (e.g., a human), such as a patient. The individual may be in need of treatment with a Sigma receptor agonists for a medical condition of any kind, including ischemic injury. Methods may be employed with respect to individuals who have tested positive for a medical condition, who have one or more symptoms of a medical condition, or who are deemed to be at risk for developing such a condition.

Sigma receptors are protein cell surface receptors and are classified as either Sigma-1 receptors or Sigma-2 receptors. In some embodiments, a Sigma receptor agonist is selected from therapeutics having pre-existing regulatory approval that are capable of being repurposed for treatment for and recovery from ischemic injury, for example, beginning during the acute phase and continuing into the chronic phase of the ischemic injury. Classes of therapeutics having pre-existing regulatory approval from which Sigma receptor agonists may be selected according to the present methods include, but are not limited to, e.g., antitussives, antipsychotics, antidepressants, neurosteroids, neuroleptics, and psychostimulants. Thus, in some embodiments, a Sigma receptor agonist comprises an antitussive, an antipsychotic, an antidepressant, a neurosteroid, a neuroleptic, or a psychostimulant, or any combination thereof. Additionally, in some embodiments, the Sigma receptor agonist is capable of crossing a subject's blood-brain barrier.

In some embodiments, the Sigma receptor is a Sigma-1 receptor (S1R). The S1R is a stress and ligand-regulated endoplasmic reticulum chaperone protein that shuttles lipids and proteins to the plasma membrane (Su, Hayashi, Maurice, Buch, & Ruoho, 2010). High densities of the S1R are found in brain tissue, including the cerebral cortex, various limbic structures, the hypothalamus, and the hippocampus (Alonso, et al., 2000; Hashimoto, Scheffel, & London, 1995). Within the nervous system, the S1R is located predominantly in the gray matter both in neurons (Alonso, et al., 2000; Klette, DeCoster, Moreton, & Tortella, 1995; Peviani, et al., 2014) and a variety of glial cell types (Gekker, et al., 2006; Hayashi & Su, 2004; Jiang, et al., 2006; Palacios, et al., 2003; Palacios, Muro, Verdu, Pumarola, & Vela, 2004; Peviani, et al., 2014; Robson, et al., 2014). In addition to modulating the actions of neurotransmitter receptors, ion channels, and synaptic function, S1Rs are involved in the regulation of diverse processes such as neuroprotection, neurorestoration, neuroplasticity, and neurotransmitter release (Kourrich, Su, Fujimoto, & Bonci, 2012; Ruscher, et al., 2012; Su, et al., 2010; Zheng, 2009).

Without wishing to be bound by theory, treatment of ischemic injury in a subject having an ischemic injury by administering an effective amount of a S1R agonist may result from the pleiotropic nature of the S1R, including not only modulation of ion channels and neurotransmitter receptors, but also intracellular calcium and endoplasmic reticulum stress, reductions in nitrosative stress, increases in brain-derived neurotropic factor (BDNF) and its receptor TrkB, and increases in antiapoptotic proteins such as Bcl2 (Ryskamp, et al., 2019). Regulation of BDNF may be particularly important as it is involved in the maintenance and repair of neurons, synaptic plasticity, and learning and memory (Cunha, Brambilla, & Thomas, 2010; Korte, et al., 1995; Lewin & Barde, 1996). In animal models, chronic administration of the S1R agonist SA4503 increases the level of BDNF protein in the rat hippocampus (Kikuchi-Utsumi & Nakaki, 2008). Similarly, in vitro SA4503 is associated with enhanced secretion of BDNF into the extracellular environment (Fujimoto, Hayashi, Urfer, Mita, & Su, 2012). S1Rs can also regulate neurite outgrowth as shown in in vitro studies using PC12 cells that demonstrated that numerous S1R ligands including (+)-pentazocine, imipramine, fluvoxamine, donepezil, SA4503, and 4-PPBP facilitate NGF-induced neurite sprouting, an effect that is reversed by S1R antagonists NE100 and BD1063 (Ishima, Fujita, & Hashimoto, 2014; Ishima & Hashimoto, 2012; Ishima, Nishimura, Iyo, & Hashimoto, 2008; Nishimura, Ishima, Iyo, & Hashimoto, 2008; Rossi, et al., 2011). However, despite promising preclinical studies, clinical trials of several S1R ligands for treating stroke (Urfer, et al., 2014), Alzheimer's disease (Schneider, et al., 2019), schizophrenia (Niitsu, et al., 2012), and peripheral neuropathy (Bruna, et al., 2018) have not been successful.

Classes of therapeutics having pre-existing regulatory approval from which S1R agonists may be selected for use according to the present methods include, but are not limited to, e.g., antitussives, antipsychotics, antidepressants, neurosteroids, neuroleptics, and psychostimulants. Thus, in some embodiments, a S1R agonist comprises an antitussive, an antipsychotic, an antidepressant, a neurosteroid, a neuroleptic, or a psychostimulant, or any combination thereof. For example, in some embodiments, the S1R agonist comprises the antipsychotic oxeladin and/or the antitussive promethazine.

The S1R agonist provided according to some of the methods described herein may be administered as part of a combination of therapeutic agents, such as a S1R agonist and at least one additional therapy, e.g., a blood pressure- and/or cholesterol-lowering medication, an anti-seizure medication, and/or one or more additional Sigma receptor agonists (e.g., 1, 2, 3, 4, 5, or more additional Sigma receptor agonists). The additional Sigma receptor agonist may be a known S1R agonist. Known S1R agonists include, but are not limited to, PRE-084, ANAVEX2-73, Donepezil, Fluvoxamine, Cutamesine, Citalopram, Amitriptyline, L-687,384, SA-4503, Dextromethorphan, Dimethyltryptamine, (+)-Pentazocine, Pentoxyverine, (+)-SKF10047, DTG, Igmesine, BD737, BD1031, BD1052, JO-1784, 3-Methoxyphencyclidine, Afobazole, Memantine, and/or Opipramol. In some embodiments, the additional therapy comprises one or more antitussives, antipsychotics, antidepressants, neurosteroids, neuroleptics, psychostimulants, or any combination thereof. In specific embodiments, the additional therapy comprises selective serotonin re-uptake inhibitors and/or ketamine.

In some embodiments, the Sigma receptor is a Sigma-2 receptor. The Sigma-2 receptor is transmembrane protein also located in the endoplasmic reticulum. It has been found to play a role in both hormone signaling and calcium signaling, in neuronal signaling, in cell proliferation and death, and in binding of antipsychotics. The Sigma-2 receptor is found in several areas of the brain, including high densities in the cerebellum, motor cortex, hippocampus, and substantia nigra, and it is also highly expressed in the lungs, liver, and kidneys. Classes of therapeutics having pre-existing regulatory approval from which Sigma-2 receptor agonists may be selected for use according to the present methods include, but are not limited to, e.g., antitussives, antipsychotics, antidepressants, neurosteroids, neuroleptics, and psychostimulants. Thus, in some embodiments, a Sigma-2 receptor agonist comprises an antitussive, an antipsychotic, an antidepressant, a neurosteroid, a neuroleptic, or a psychostimulant, or any combination thereof.

The Sigma-2 receptor agonist provided according to some of the methods described herein may be administered as part of a combination of therapeutic agents, such as a Sigma-2 receptor agonist and at least one additional therapy, e.g., a blood pressure- and/or cholesterol-lowering medication, an anti-seizure medication, an antitussive, an antipsychotic, an antidepressant, a neurosteroid, a neuroleptic, a psychostimulant, and/or one or more additional Sigma receptor agonists (e.g., 1, 2, 3, 4, 5, or more additional Sigma receptor agonists). In some embodiments, the additional therapy is ketamine. In some embodiments, the additional therapy is a selective serotonin re-uptake inhibitor. The additional Sigma receptor agonist may be a known Sigma-2 receptor agonist. Known Sigma-2 receptor agonists include, but are not limited to, CB 64D, CB-184, RC-106, DTG, PB28, Siramesine, SV119, F281, and/or WC-26.

### C. Methods of Use

Aspects of the present disclosure are directed to methods comprising treatment of a subject suffering from, or suspected of having, an ischemic injury. In some embodiments, the present disclosure provides methods for ischemic injury treatment that employs one or more Sigma receptor agonists, comprising administering an effective amount of a Sigma receptor agonist of the present disclosure. In one embodiment, methods are encompassed herein for treating, delaying progression of, delaying onset of, or reducing the risk of ischemic injury in an individual by administering to the individual an effective amount of the Sigma receptor agonist to treat the ischemic injury in the subject. The present methods may be applied for the treatment of ischemic injury induced by a cerebral ischemic. In specific embodiments, the cerebral ischemia is ischemic stroke. In specific embodiments, the cerebral ischemia is thrombotic ischemia, embolic ischemia, or hypoperfusion, and the cerebral ischemia may result in ischemic stroke. The ischemic injury may be of any type and of any phase, e.g., hyper-acute, acute, subacute, and/or chronic. The individual may have an ischemic injury or be at risk for ischemic injury, including over the general population. A subject may be identified as having an ischemic injury or being at risk for ischemic injury using tests and diagnostic methods known in the art.

In some embodiments, the disclosed methods comprise treating a subject suffering from an ischemic injury. As disclosed herein, ischemic injuries, especially during the acute and continuing into the chronic phase of the injuries, are surprisingly and unexpectedly sensitive to treatment with Sigma receptor agonists. Further, administering a Sigma receptor agonist was surprisingly found to stimulate release of brain-derived neurotropic factor from neurons and to reduce one or more neurological deficits and/or symptoms. Accordingly, in some embodiments, disclosed is a method of treating ischemic injury in a subject having an ischemic injury, the method comprising administering to the subject a composition comprising an effective amount of a Sigma receptor agonist. The ischemic injury can be of any type and/or due to any cause, but in some embodiments, the ischemic injury is induced by a cerebral ischemia. The cerebral ischemia can be thrombotic ischemia, embolic ischemia, or hypoperfusion, and in specific embodiments, the cerebral ischemia is ischemic stroke. Thus, in some embodiments, disclosed is a method of treating ischemic stroke by administering a composition comprising an effective amount of a Sigma receptor agonist.

In particular embodiments, the pharmaceutical compositions of the present disclosure may be particularly useful in ameliorating and/or treating ischemic injuries induced by cerebral ischemia, including thrombotic ischemia, embolic ischemia, or hypoperfusion, and in specific cases, ischemic stroke. In some embodiments, the ischemic injury is an ischemic injury characterized as having hyper-acute, acute, subacute, and/or chronic phases. In some embodiments, the composition is administered during the hyper-acute phase of the ischemic injury. For example, in some embodiments, the composition is administered within at least about 24 hours after occurrence of the ischemic injury (e.g., within about 0.1, 0.5, 1, 2, 3, 4, 5, 6, 12, or 24 hours, or any range or value derivable there between, after occurrence of the ischemic injury). In some embodiments, the composition is administered during the acute phase of the ischemic injury. For example, in some embodiments, the composition is administered within at least about 7 days after occurrence of the ischemic injury (e.g., within about 0.1, 0.5, 1, 2, 3, 4, 5, 6, 12, 24, 36, 48, 72, 96, 120, 144, or 168 hours, or any range or value derivable there between, after occurrence of the ischemic injury). In some embodiments, the composition is administered during the subacute phase of the ischemic injury. For example, in some embodiments, the composition is administered within at least about 3 weeks after occurrence of the ischemic injury (e.g., within about 1, 2, 3, 4, 5, 6, 7, 8, 9, 10, 11, 12, 13, 14, 15, 16, 17, 18, 19, 20, or 21 days, or any range or value derivable there between, after occurrence of the ischemic injury). Additionally or alternatively, in some embodiments, the composition is administered during the chronic phase of the ischemic injury, e.g., more than 3 weeks after occurrence of the ischemic injury.

In specific embodiments, the composition is administered within at least about 6 hours after occurrence of the ischemic injury. In specific embodiments, the composition is administered within at least about 12 hours after occurrence of the ischemic injury. In specific embodiments, the composition is administered within at least about 24 hours after occurrence of the ischemic injury. In specific embodiments, the composition is administered within at least about 48 hours after occurrence of the ischemic injury.

In specific embodiments, the composition is administered at least about 6 hours after occurrence of the ischemic injury (e.g., about 6, 12, 24, 36, 48, 60, 72, 84, or 96 hours, or any range or value derivable there between, after occurrence of the ischemic injury). In some embodiments, the composition is administered preferably 12 hours, more preferably 24 hours, still more preferably 36 hours, or even more preferably 48 hours after incidence of the ischemic injury.

Thus, in specific cases, examples of treatment methods are as follows: (1) therapy with a Sigma receptor agonist to treat subjects with any type of ischemic injury during any acute and/or chronic phase of the ischemic injury; (2) therapy with a Sigma receptor agonist to treat subjects with an ischemic injury induced by a cerebral ischemia during any acute and/or chronic phase of the ischemic injury; (3) therapy with a Sigma receptor agonist to treat subjects with an ischemic injury induced by a thrombotic ischemia during any acute and/or chronic phase of the ischemic injury; (4) therapy with a Sigma receptor agonist to treat subjects with an ischemic injury induced by an embolic ischemia during any acute and/or chronic phase of the ischemic injury; (5) therapy with a Sigma receptor agonist to treat subjects an ischemic injury induced by hypoperfusion during any acute and/or chronic phase of the ischemic injury; and/or (6) therapy with a Sigma receptor agonist to treat subjects an ischemic injury induced by ischemic stroke during any acute and/or chronic phase of the ischemic injury.

Sigma receptor agonists, as contemplated herein, and/or pharmaceutical compositions comprising the same, can be administered alone or in any combination, and in at least some aspects, together with a pharmaceutically acceptable carrier or excipient, and can be used for the treatment and/or amelioration of ischemic injury. In some embodiments, the Sigma receptor agonist comprises a therapeutic having pre-existing regulatory approval that is repurposed according to the present methods for treatment for and/or recovery from ischemic injury and that can cross a subject's blood-brain barrier. For example, in some embodiments, the Sigma receptor agonists may be an antitussive, an antipsychotic, an antidepressant, a neurosteroid, a neuroleptic, or a psychostimulant, or any combination thereof. In specific embodiments, the Sigma receptor agonists is an antitussive, and the antitussive is promethazine. In specific embodiments, Sigma receptor agonists is an antipsychotic, and the antipsychotic is oxeladin.

In some embodiments, the disclosed methods comprise identifying one or more subjects as being candidates for treatment with a Sigma receptor agonist, based on current or prior symptoms of ischemic injury and/or ischemic injury phase. For example, in some embodiments, disclosed is a method comprising identifying a subject having an ischemic injury as being a candidate for treatment with a Sigma receptor agonist by determining that the subject currently has or previously had symptoms of ischemic injury. Additionally or alternatively, in some embodiments, a subject having an ischemic injury is identified as being a candidate for treatment with a Sigma receptor agonist by determining that the subject is past the hyper-acute phase of the ischemic injury and is in the acute, subacute, and/or chronic phase of the ischemic injury. In some embodiments, the disclosed methods comprise determining an optimal treatment for a subject with symptoms of ischemic injury in the acute phase and continuing into the chronic phase of the ischemic injury. In some embodiments, a subject is given multiple types of therapy, for example, a combination of therapeutic agents, such as a S1R agonist and at least one additional therapy, e.g., a blood pressure- and/or cholesterol-lowering medication, an anti-seizure medication, antitussives, antipsychotics, antidepressants, neurosteroids, neuroleptics, psychostimulants, and/or one or more additional Sigma receptor agonists (e.g., 1, 2, 3, 4, 5, or more additional Sigma receptor agonists).

Subjects treated with the present Sigma agonist therapy may or may not have been treated for the ischemic injury prior to receiving the Sigma agonist therapy. In some embodiments, the patient has received at least 1, 2, 3, 4, 5, 6, 7, 8, or more prior treatments for ischemic injury, e.g., during a hyper-acute, acute, and/or subacute phase of the ischemic injury and/or during a chronic phase of the ischemic injury. The prior treatments may include a treatment or therapy described herein. In some embodiments, the prior treatments comprise drugs to restore blood flow to the brain, emergency surgical endovascular procedures, blood pressure- and/or cholesterol-lowering medication, anti-seizure medication, antitussives, antipsychotics, antidepressants, neurosteroids, neuroleptics, psychostimulants, and/or one or more additional Sigma receptor agonists (e.g., 1, 2, 3, 4, 5, or more additional Sigma receptor agonists), and the like. In some embodiments, the subject had received the prior therapy within 0.1, 0.25, 0.5, 0.75, 1, 2, 3, 4, 5, 6, 7, 8, 9, 10, 15, 20, 25, 30, 35, 40, 45, 50, 55, or 60 days, hours, or weeks, or any range or value derivable therein, of administration of the current compositions of the disclosure. The subject may therefore utilize the treatment method of the disclosure as an initial treatment or after (and/or with) another treatment. In some embodiments, the methods may be tailored to the need of a subject with an ischemic injury based on the type and/or stage of the injury, and in at least some cases, the therapy may be modified during the course of treatment for the subject.

### D. Compositions

In some embodiments, pharmaceutical compositions (e.g., compositions comprising a Sigma receptor agonist) are administered to a subject. Different aspects may involve administering an effective amount of a composition (e.g., compositions comprising a Sigma receptor agonist) to a subject. In some embodiments, a Sigma receptor agonist may be administered to the subject to treat or ameliorate a condition (e.g., ischemic injury). Additionally, such compositions can be administered in combination with one or more additional therapeutic agents (e.g., an additional Sigma receptor agonist, a blood pressure-lowering medication, an anticonvulsant, etc.). The different therapies may be administered in one composition or in more than one composition, such as 2 compositions, 3 compositions, or 4 compositions. In some embodiments, the Sigma receptor agonist and one or more additional therapies are administered in separate compositions. In some embodiments, the Sigma receptor agonist and one or more additional therapies are in the same composition. Various combinations of the agents may be employed. The therapeutic agents of the disclosure, including Sigma receptor agonists and additional therapeutics, may be administered by the same route of administration or by different routes of administration.

Also contemplated are methods of producing a composition suitable for treating an ischemic injury in a subject. Such methods may comprise providing an effective amount of a Sigma receptor agonist capable of treating the ischemic injury; and combining the Sigma receptor agonist with one or more additional therapeutic agents and/or one or more pharmaceutically acceptable excipients to obtain the composition.

The pharmaceutical compositions may include various “unit doses.” Unit dose is defined as containing a predetermined-quantity of the therapeutic composition. The quantity to be administered, both according to number of treatments and unit dose, depends on the treatment effect desired. An effective dose is understood to refer to an amount necessary to achieve a particular effect. Effective amounts of Sigma receptor agonist, or a composition thereof, can be administered in treatment regimens consistent with the ischemic injury, for example a single or a few doses over one to several days to ameliorate one or more symptoms or periodic doses over an extended time to inhibit injury progression and prevent injury recurrence. The precise dose to be employed in the formulation will also depend on the type of disease to be treated, severity and course of the disease, the clinical condition of the individual, the individual's clinical history and response to the treatment, the manner of administration, the intended goal of treatment (alleviation of symptoms versus cure), and/or the potency, stability and toxicity of the particular therapeutic substance or other therapies a subject may be undergoing and should be decided according to the judgment of the practitioner and each subject's circumstances.

In specific embodiments, the dosing regimen is a single dose of an effective amount of the Sigma receptor agonist, or a composition thereof. In other embodiments, the subject is provided with multiple doses of an effective amount of the Sigma receptor agonist, or a composition thereof. In cases where the subject is provided with two or more doses of an effective amount of the Sigma receptor agonist, or a composition thereof, the duration between doses is 1, 2, 3, 4, 5, 6, 7, 8, 9, 10, 11, 12 or more hours, 1, 2, 3, 4, 5, 6, 7 or more days, or 1, 2, 3, or 4 or more weeks, or 1, 2, 3, 4, 5, 6, 7, 8, 9, 10, 11, 12, or more months. In specific embodiments, the subject is provided with one or more doses of an effective amount of the Sigma receptor agonist, or a composition thereof, daily.

In some embodiments, an effective amount of the Sigma receptor agonist, or a composition thereof, is administered to stimulate release of brain-derived neurotropic factor from neurons. In some embodiments, the Sigma receptor agonist, or a composition thereof, is administered to reduce one or more neurological deficits and/or symptoms. In some embodiments, the Sigma receptor agonist, or a composition thereof, is administered at least until one or more symptoms of the ischemic injury, e.g., hemiparesis, hypesthesia, hemianopsia, aphasia, speech disinhibition, dysarthria, agnosia, cortical sensory deficits, impaired or altered mental status or memory, incontinence, gait apraxia, nystagmus, diploia, vertigo, dysphagia, syncope, and/or ataxia, are resolved.

Therapeutically effective amounts of the Sigma receptor agonist, or a composition thereof, can be administered by a number of routes, including, but not limited to, for example, orally, topically, transdermally, subcutaneously, intravenously, intraperitoneally, intramuscularly, intrathecally, intraventricularly, intranasally, or by infusion. The agents in some aspects of the disclosure may be formulated into preparations for local delivery (i.e. to a specific location of the body) or systemic delivery, in solid, semi-solid, gel, liquid or gaseous forms such as tablets, capsules, powders, granules, ointments, solutions, depositories, inhalants and injections allowing for oral, parenteral or surgical administration. The therapeutically effective amount of the Sigma receptor agonist, or a composition thereof, for use in a method of treating ischemic injury is that amount that achieves a desired effect in a subject being treated. For instance, this can be the amount of Sigma receptor agonist necessary to inhibit advancement, or to cause regression of, ischemic injury, or which is capable of relieving symptoms caused by ischemic injury.

In the practice of certain embodiments, it is contemplated that doses in the range from 10 mg/kg to 200 mg/kg can affect the therapeutic capability of Sigma receptor agonists. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000, 2000, 3000, 4000, or 5000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

Pharmaceutical compositions and formulations as described herein can be prepared by mixing the active ingredients (such as a Sigma receptor agonist) having the desired degree of purity with one or more optional pharmaceutically acceptable carriers (Remington's Pharmaceutical Sciences 22nd edition, 2012), in the form of lyophilized formulations or aqueous solutions. Thus, the therapeutic agents of the disclosure, or compositions thereof, will generally be dissolved or dispersed in a pharmaceutically acceptable carrier or aqueous medium. The carrier is non-toxic, biocompatible, and is selected so as not to detrimentally affect the biological activity of the agent.

Other pharmaceutically acceptable carriers include non-aqueous and aqueous solutions, non-toxic excipients, including salts, preservatives, buffers and the like. Examples of pharmaceutically acceptable carriers include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); anti-bacterial and anti-fungal agents (such as parabens; chlorobutanol; phenol; sorbic acid; and thimerosal) low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn— protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

The compositions may be formulated into a neutral or salt form. Pharmaceutically acceptable salts, include the acid addition of salts (formed with the free amino groups of the protein) and are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

In certain aspects, the pharmaceutical compositions are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste, or salve. For instance, the composition may contain 10 mg or less, 25 mg, 50 mg or up to about 100 mg of human serum albumin per milliliter of phosphate buffered saline.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

Additional formulations are suitable for intranasal administration or administration by inhalation. Aerosol delivery can be used for these formulations. Volume of the aerosol may be between about 0.01 ml and 0.5 ml, for example.

Compositions according to the present invention can be prepared according to standard techniques and may comprise water, buffered water, saline, glycine, dextrose, iso-osmotic sucrose solutions and the like, including glycoproteins for enhanced stability, such as albumin, lipoprotein, globulin, and the like. These compositions may be sterilized by conventional, well-known sterilization techniques. The resulting aqueous solutions may be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, and the like. The preparation of compositions that contains the Sigma receptor agonists will be known to those of skill in the art in light of the present disclosure, as exemplified by Remington: The Science and Practice of Pharmacy, 21st Ed. Lippincott Williams and Wilkins, 2005, incorporated herein by reference. Moreover, for animal (e.g., human) administration, it will be understood that preparations should meet sterility, pyrogenicity, general safety, and purity standards as required by FDA Office of Biological Standards.

### E. Kits

Kits are also contemplated as being used in certain aspects of the present invention. For instance, a composition of the present invention can be included in a kit, and the compositions may be suitably aliquoted. The composition may comprise a Sigma receptor agonist, one or more additional therapeutic agents, and/or one or more pharmaceutically acceptable excipients. The kit may further include other materials desirable from a commercial and user standpoint, including other buffers, diluents, filters, needles, syringes, and package inserts with instructions for use.

A kit can include a container. Containers can include a bottle, a vial, a tube, a flask, a bag, a syringe, a metal tube, a laminate tube, a plastic tube, a dispenser, a pressurized container, a barrier container, a package, a compartment, or other types of containers such as injection or blow-molded plastic containers into which the compositions or desired bottles, dispensers, or packages are retained. Where there are multiple components in the kit, the kit also may contain a second, third, or other additional container into which the additional components may be separately placed. The container(s) may be formed from a variety of materials such as glass, plastic (such as polyvinyl chloride or polyolefin), or metal alloy (such as stainless steel or a nickel-molybdenum alloy). The kit and/or container(s) can include indicia on its surface. The indicia, for example, can be a word, a phrase, an abbreviation, a picture, or a symbol.

The container(s) can dispense a pre-determined amount of a composition. In other embodiments, the container(s) can be squeezed (e.g., metal, laminate, or plastic tube) to dispense a desired amount of the composition. The composition can be dispensed as, e.g., a tablet, a spray, a foam, an aerosol, a liquid, a fluid, or a semi-solid. The container(s) can have spray, pump, or squeeze mechanisms. A kit can also include instructions for using the kit and/or compositions to treat ischemic injury. Instructions can include an explanation of how to administer, apply, use, and/or maintain the compositions.

### EXAMPLES

As part of the disclosure of the present invention, specific examples are included below. The examples are for illustrative purposes only and are not intended to limit the invention in any manner. Those of ordinary skill in the art will readily recognize a variety of noncritical parameters, which can be changed or modified to yield essentially the same results.

### Example 1

### Materials and Methods for Screening & Testing Sigma Receptor Agonists

Chemicals and reagents. Compounds were purchased from the following sources: 4-PPBP, BD1063, (+)-igmesine, L-741,742, and L-745,870 were from Tocris Biosciences (Minneapolis, MN); butamirate, carbetapentane, and NE-100 were from Santa Cruz Biotechnology (Dallas, TX); and donepezil oxeladin, promethazine, haloperidol metabolite II, and oxeladin citrate were from Sigma-Aldrich (St. Louis, MO). Radioligands were purchased from PerkinElmer: ([³H]-(+)-pentazocine ((+)-Pentazocine, [RING-1,3-³H], 33.9 Ci/mmol, NET1056), [³H]DTG (1,3-Di-o-tolylguanidine, [p-RING-³H]-, 50 Ci/mmol, NET986) (Saint Louis, MO). All compounds were prepared in DMSO at concentrations ranging from 10-100 mM. Stocks were then diluted 1:1000 (v/v) in the final assay solution.

Cell culture. Human MCF-7 cells (American Type Cell Culture, Manassas, VA) were grown in Dulbecco's Modified Eagle's Medium (DMEM; Fisher Scientific, Pittsburgh, PA) supplemented with 10% Fetal Bovine Serum (FBS, Fisher Scientific, Pittsburgh, PA), 100 g/ml nonessential amino acids (Hyclone, Logan, UT), 2 mM L-glutamine (Sigma-Aldrich, St. Louis, MO), and 10 g/L Bovine Insulin (Sigma-Aldrich, Sigma-Aldrich, St. Louis, MO). MCF-7 cells served as the source of human Sigma-2 receptors (S2R) as they express the S2R but lack detectable levels of the S1R (Schetz, et al., 2007; Vilner, John, et al., 1995). MCF-7 cells stably expressing the human S1R (MCF7-hS1R) were prepared as described previously (Schetz, et al., 2007) and kept under constant selective pressure with 100 μg/mL G-418 (Invitrogen, San Diego, CA). Rat PC12 cells were purchased from American Type Culture Collection (ATCC, Manassas, VA) and PC-6-15 cells, a variant of the PC12 cells overexpressing the trkA receptor (Hempstead, et al., 1992), were obtained Dr. Moses Chao. Both PC12 and PC-6-15 cells were maintained in RPMI 1640 media supplemented with 10% horse serum and 5% FBS. The PC-6-15 cells were grown under constant selective pressure with 100 μg/mL G-418. MN9D cells were obtained from ATCC and were maintained in DMEM complete with 10% FBS. All culture media were additionally supplemented by 100 IU/mL of penicillin and streptomycin (Corning, Manassas, VA) and 1 mM sodium pyruvate (Sigma-Aldrich, St. Louis, MO), and grown at 37° C. under 95% air, 95% humidity, and 5% CO₂.

Animals and housing. Adult male Sprague-Dawley rats (200-250 g) were obtained from ENVIGO and housed under controlled temperature and lighting conditions (lights on 0700-1900 h). Rats were pair housed and fed ad libitum with standard laboratory chow and free access to water. Rats were allowed at least 1 week to acclimate to the animal facility prior to beginning studies.

Measuring receptor density with radioligand binding. The density or maximum number of binding sites (Bₘₐₓ) for the S1R in PC-6-15 cells was estimated by employing 10 nM of [³H]-(+)-pentazocine as the radioligand followed by calculation of the Bₘₐₓ at saturation using a square hyperbola model: Bₘₐₓ=(Y·(K_{D}+X))/X, where Y is [specifically bound radioligand] and X=[radioligand concentration]. The affinity (K_{D}) for [³H]-(+)-pentazocine at the cloned human S1R had been previously determined to be 3.7 nM (Schetz, et al., 2007).

Measuring the ligand affinities at SIR and S2R receptors by radioligand binding. The affinity (Kᵢ) values of compounds interacting with the S1R and S2R were determined by displacement of 0.5 nM [³H]-(+)-pentazocine from MCF-7-S1R and 2.5 nM [³H]DTG from untransfected MCF-7 cells, respectively. Binding conditions were the same as described previously for the S1R (Schetz, et al., 2007): binding buffer (Tris 50 mM, pH=8.1 at 37° C.), ice-cold wash buffer (Tris 10 mM, pH=8.1 at 0-2° C.), and incubation time (3 hrs at 37° C.) with shaking. Non-specific binding for the S1R and S2R was determined in the presence of 5 μM BD1063 and 15 M haloperidol, respectively. Following incubation, receptors were collected via rapid filtration through GF/C filters (Brandel, Gaithersburg, MD) followed by washing three times with 3 mL of ice-cold wash buffer. Dried filters were transferred to vials filled with 3.5 ml of scintillation fluid, and the radioactivity was quantified on a liquid scintillation analyzer (TRICARB® 2800TR) from Perkin Elmer (Saint Louis, MO). Mean values from duplicate or triplicate determinations are reported along with their associated standard error of the mean (SEM). Kᵢ values were calculated from IC₅₀ values using the Cheng-Prusoff equation. The concentration of membrane protein was determined using a PIERCE® BCA Protein Assay kit (Life Technologies, Grand Island, NY) following the manufacturer's protocol.

Measuring BDNF secretion via in situ ELISA. The in situ ELISA format utilized herein has been shown to have improved sensitivity related to its rapid capture of secreted BDNF (Balkowiec & Katz, 2000; Dalwadi, Kim, & Schetz, 2017). The in situ ELISA improves on traditional ELISA by seeding BDNF-secreting cells directly into the wells pre-coated with an anti-BDNF primary antibody. BDNF secreted by these cells is immediately captured by the primary antibody, drastically increasing the sensitivity and reproducibility of the ELISA assay. Intact cells are removed subsequent to the addition of the secondary and tertiary antibodies. The amount of BDNF secreted from the neuronal MN9D cells was quantified using an in situ ELISA assay developed using the BDNF EMAX® ImmunoAssay kit (Cat. No. G7611, Promega, Madison, WI). Briefly, a NUNC™ MAXISORP™ flat-bottom, polystyrene, 96-well immunoplate was coated for 48 hrs at 4° C. with an anti-BDNF monoclonal antibody diluted 1:1000 v/v in carbonate buffer containing 25 mM sodium bicarbonate and 25 mM sodium carbonate, pH 9.7. Unbound antibody was removed by washing 5 times with 150 μL of TBST buffer (20 mM Tris-HCl, pH7.6, 150 mM NaCl, and 0.05% (v/v) TWEEN® 20), before blocking non-specific sites first with blocking buffer for 1 hr and then with culture medium for 2 hrs. Cells were seeded at 35,000 cells per well and incubated overnight at 37° C. in a humidified CO₂ incubator. The following day, the wells were replaced with fresh culture media containing either experimental compounds or vehicle controls, then incubated for an additional 24 hours. In addition, on the same plate, but in separate wells, BDNF standards were added ranging in concentration from 15.6-250 pg/mL. After 24 hrs incubation with experimental compounds, the media was aspirated and 100 μL of Dulbecco's phosphate saline (D-PBS without Ca²⁺ and Mg²⁺ supplemented with 5 mM EDTA) was added to each well and incubated for 15 min at 37° C. to promote cell lifting. Cells were then detached from the bottom of wells by triturating in the center and around the edges of the well. After removing all cell debris, the wells were rinsed five times with 150 μL of TBST. The plate was then incubated with 1:500 v/v diluted polyclonal anti-human BDNF antibody for 2 hrs at room temperature. This antibody was removed, and wells were washed five times with 150 μL of TBST, before incubating with 1:200 v/v diluted polyclonal Anti-IgY HRP conjugate for 2 hrs at room temperature. Wells were then washed five times with 150 μL of TBST and the remaining specifically bound polyclonal antibody was detected with the 50 μL colorimetric HRP substrate 3,3′,5,5′-Tetramethylbenzidine (TMB). The reaction was terminated with 50 μL of 1 M HCl and the color intensity was quantified by measuring the absorbance at 450 nm using a FLEXSTATION® 3 plate reader (Molecular Devices, Sunnyvale, CA). Measurements from multiple experiments were normalized to maximal BDNF responses achieved by stimulating the prototypical sigma ligand 4-PPBP. A one-way ANOVA with a Bonferroni multiple comparisons post-hoc analysis (P<0.05) was applied to determine significant differences between groups. When converted to pg/mL averaged BDNF values±SEM (n=3-10 experiments) were: 75.4±6.9 for baseline (vehicle control) and 128.6±12.8 for maximal stimulation by 10 μM 4-PPBP.

Measuring the maximum tolerated dose of Sigma receptor agonists. Male Sprague-Dawley rats (2-3 per dose) were treated by oral gavage with 6, 18, 54, 108, 135 or 150 mg/kg as estimated from in vitro functional assays and estimates of human intake when used as an antitussive. Rats received a single daily dose for 10 days. Weight, water intake, and food intake were measured daily, and rats were assessed for somnolence, activity, sensitivity to touch, and taste aversion. At the conclusion of the study, rats were humanely euthanized and brain tissue was collected and flash frozen.

Evaluating the pharmacokinetics of Sigma receptor agonists. Twenty-one adult male Sprague Dawley rats (n=3/time point) were dosed PO with 135 mg/kg oxeladin citrate, 0.5 ml/rat in 0.9% normal saline. Whole blood was harvested into EDTA coated tubes at 30, 60, 120, 240, 720, and 1440 minutes from rats following euthanasia by CO₂ inhalation. Plasma was processed from whole blood by centrifugation for 10 min at 10,000 rpm and frozen at −80° C. until assayed. The brains were collected, weighed, snap frozen in liquid nitrogen, and stored at −80° C. until processed.

For standards, 98 μl blank plasma was added to an Eppendorf tube spiked with 2 μl of initial standard (IS). For QCs, add 98.8 μl blank plasma was added to an Eppendorf tube spiked with 1.2 μl of IS. 100 μl plasma was mixed with 200 μl of acetonitrile containing 0.15% formic acid and 37.5 ng/ml IS (IS final conc.=25 ng/ml), and 100 microliters brain homogenized solution was mixed with 200 microliters of acetonitrile containing 0.15% formic acid and 60 ng/ml IS (IS final conc.=40 ng/ml). The samples were vortexed 15 sec, incubated at room temp for 10 min and spun 2×13,200 rpm in a standard microcentrifuge. The supernatant was then analyzed by LC/MS/MS (QTRAP® 3200) according to the following parameters:

Ion Source/Gas Parameters: CUR=25, CAD=medium, IS=5000, TEM=700, GS1=70, GS2=70.

Buffer A: Water+0.1% formic acid; Buffer B: ACN+0.1% formic acid; flow rate 1.5 ml/min; column Agilent Eclipse XDB-C18 column, 5 micron packing 50×4.6 mm size; 0-1.5 min 3% B, 1.5-2.0 min gradient to 100% B, 2.0-3.5 min 100% B, 3.5-3.6 min gradient to 3% B, 3.6-4.5 3% B. IS: n-benzylbenzamide (Sigma-Aldrich, lot #02914LH, in ACN, transition 212.1 to 91.1); Compound transition 336.127/100.1.

Clotting assays. Thrombotic potential was assessed with trunk blood from donor rats using a protocol modified from Kim et al. (Kim et al., 2010). Oxeladin was compared to whole blood with or without the anticoagulant benzamidine and several other S1R ligands at doses 10× greater than those that stimulate the release of BDNF (Dalwadi et al., 2022; Dalwadi et al., 2017). Blood was rapidly collected into chilled, pre-weighed plastic tubes containing a known quality of EDTA disodium salt (>3.7 mM) electrostatically-coated onto the tube wall. Blood was prevented from coagulating by gently swirling the chilled EDTA-coated tube. The tube was weighed and the volume of collected blood calculated in order to compute the exact concentration of EDTA in the uncoagulated blood sample allowing us to determine which concentration of calcium to add back into the blood to initiate coagulation. Uncoagulated blood samples were stored chilled. The following was be dispensed into NUNC™ MICROWELL™ MiniTrays: 8 μL/well of normal saline containing a concentration of calcium 1.2 mM higher than that of the concentration of EDTA in the blood sample, 1 μL/well of oxeladin, 3 mM benzamidine anticoagulant positive control, or untreated or vehicle negative controls, and 1 μL/well of (EDTA-treated) uncoagulated rat blood. Plates were tilted at a 45° angle at 5, 15, 30, 45 and 60 minutes to observe coagulation (i.e., no streaking) and compared to the controls. Each triplicate well was scored by a treatment-blinded observer as Yes (coagulation, no streaking) or No (no coagulation, streaking). The experiment was repeated with two separate donor rats.

Evaluating the effect of Sigma receptor agonists in a rat model of focal cerebral ischemia. Rats were anesthetized with 5% isoflurane in oxygen and maintained at ˜2% through a nosecone. Core temperature was maintained with an infrared heating pad controlled by a rectal probe. Transient middle cerebral artery occlusion (tMCAO) was performed as previously described (Oppong-Gyebi et al., 2022). Briefly, a midline incision was made from the neck to the sternum. The left common carotid artery and bifurcation was mobilized, and a silicon-coated suture (Doccol, Sharon MA) was inserted through the external carotid artery into the internal carotid artery to reach the origin of the MCA at the base of the brain. The incision was closed, and rats were allowed to awaken so that successful occlusion could be assessed. Rats not displaying hemiparesis immediately post-surgery were excluded from the study. After 85-90 minutes occlusion, rats were anesthetized, and the suture was withdrawn to allow reperfusion. Rats were injected subcutaneously with 5 ml saline bilaterally to provide hydration during recovery. On the day following tMCAO, rats were assessed for persistent signs of stroke.

Only rats displaying hemiparesis immediately post-surgery were included in the study. Rats with a Bederson score of 0 or <50% contralateral bias on the elevated body swing test (EBST) (Borlongan and Sanberg, 1995) on Day 1 after tMCAO were excluded from the study. Animals that became moribund or died within 24 hours of tMCAO were excluded from the study.

Two days before tMCAO rats were trained to lick 5% sucrose from a 1 ml syringe. After behavioral assessment on Day 1 after tMCAO, rats were randomly assigned to 4 groups in a blinded fashion. Groups included sucrose Vehicle, or 45, 68 or 135 mg/kg oxeladin. Oxeladin was prepared in 5% sucrose and provided to rats from the end of a 1 ml syringe. Starting 48 hours after stroke, rats were administered treatments daily from day 3 to day 14. On days 3-14 rats were injected i.p. with 50 mg/kg bromodeoxyuridine (BrdU; Fisher Bioreagents). At the conclusion of the study, rats were euthanized under deep isoflurane anesthesia. Rats were transcardially perfused with cold saline and either decapitated for brain collection and flash freezing of brain tissue or transcardially perfused with 4% formaldehyde for immunohistochemical studies.

On days 1, 3, 7, and 14 after tMCAO, rats underwent a series of behavioral tests by a treatment blinded observer. All tests were performed in a room dimly lit with red light. Rats were assessed by the Bederson test (Bederson et al., 1986), and an 11-point Neurological Deficit test as previously described (Oppong-Gyebi et al., 2022). The elevated body swing test (EBST) was performed in an open field with 30 seconds between each of 20 lifts by the base of the tail (Borlongan and Sanberg, 1995). The EBST was scored as percent of nose swing to the contralateral side to the tMCAO.

Assessment of infarct and inflammation. For immunohistochemical assessments 6 random animals were chosen from each group by a treatment-blinded observer to reduce the chance of bias. Nissl staining was performed using thionin. Rats were deeply anesthetized with isoflurane and transcardially perfused with saline and 4% formaldehyde. Brains were removed and post fixed for 48 hours. Coronal sections were made at 40 m through the region of the middle cerebral artery, affixed to gelatin-coated glass slides and stained. Four sections representing Bregma levels ˜0.7, 0.2, −0.8, and −2.12 were assessed for each animal. Sections were scanned with a 4× objective using a Keyence BZ-X800 microscope and analyzed using Image J. Infarct assessment was performed by a treatment blinded observer two separate times and the average infarct was assessed by determining the extent of histologically healthy tissue using the method of Swanson (Swanson et al., 1990).

The inflammatory response to stroke was observed using immunohistochemistry for glial fibrillary acidic protein (GFAP, Neomarkers RB-087, 1:5000) to assess astrogliosis and ionized calcium binding adapter molecule 1 (Iba1 Fujifilm Wako #019-19741, 1:5000), followed by a biotinylated secondary antibody (Jackson Immuno Research #711-065-152, 1:400). Immunostaining was revealed using Vector Labs VECTASTAIN® ELITE® ABC (PK-6100) and IMMPACT® DAB (SK-4105) kits. Area of staining the striatum, cortex, and dorsal hippocampus was assessed by a treatment-blinded observer using wide-field images captured with a 4× objective using a Keyence BZ-X800. The percent area above threshold was determined with Image J. Images were processed by a treatment-blinded observer using ImageJ by converting images to 8 bits, manually outlining the areas of interest using the corpus callosum to define the boundaries of the striatum and cortex, subtracting background and subjecting the images to auto thresholding. When more than one staining run was required to process tissue, the mean gray values of a standard ROI in the contralateral cortex were used to normalize between the runs.

Actions of oxeladin on BDNF in the brain. To assess effects of oxeladin on steady-state BDNF concentrations in the brain, rats from the maximum tolerated dose study (above) were used to assay BDNF in tissue homogenates from vehicle (n=2) and 135 mg/kg oxeladin treatment (n=5) by ELISA using the Promega BDNF EMAX® ImmunoAssay system. Cerebral cortex and hippocampal samples were homogenized in TBS supplemented with 0.1% NP-40 detergent and protease inhibitor cocktail. Protein concentrations were determined with a PIERCE® BCA assay (ThermoPierce). Samples were diluted 1:20 and assayed in triplicate according to manufacturer's instructions and assayed a FLEXSTATION® 3 plate reader (Molecular Devices, Sunnyvale, CA).

To assess effects of oxeladin action in the rat brain after stroke, a separate cohort of animals (n=12/gp) was subjected to tMCAO and given a single dose of oxeladin (135 mg/kg) 48 hours later. Rats were sacrificed under isoflurane anesthesia 2, 6, or 24 hours after dosing, and brains were removed to assess protein expression in the ischemic and contralateral cortex by immunoblotting. Brains were rapidly removed, chilled for 2 min in ice-cold saline, and sliced into 2 mm coronal slices with a brain matrix for rapid 2,3,5 triphenyltetrazolium chloride (TTC) staining. Sections were immersed in 1% TTC for 1 minute at room temperature to reveal the extent of the infarct. Cortical penumbra tissue was collected, snap frozen, and stored at −80° C. for subsequent western blotting. Protein was extracted from tissue using T-PER™ reagent with HALT protease inhibitor (Thermo-Pierce), and concentrations were determined with the PIERCE® BCA assay (Thermo-Pierce). Protein was separated on 4-20% SDS-PAGE gels (BioRad) and transferred to nitrocellulose. Blots were probed with primary antibodies for BDNF (Abcam ab226842; 1:1000) and goat anti-rabbit-HRP secondary (Thermo 32260; 1:10,000). Bands were visualized by ECL with PIERCE® SUPERSIGNAL® West Dura Femto (Thermo-Pierce) reagent using a UVP EpiChemi3. Density was assessed with Image J and normalized to β-actin (Thermo MA1-91399; 1:1000).

Actions of oxeladin on proliferation in the brain. Fourteen days after tMCAO, rats were transcardially perfused with saline and 4% formaldehyde, as described above. Brains were removed and post-fixed for 48 hours in 4% formaldehyde and then moved to phosphate buffer. Brains were blocked and serial 30 mm coronal sections through the subventricular zone (SVZ) and dorsal hippocampus (HP) were made with a cryostat. Sections were affixed to glass slides and stored at −20° C. until processed. Sections were subjected to antigen retrieval with 10 mM sodium citrate (pH 6.0). Slides were immersed in preheated buffer, placed in a commercial vegetable steamer at 99° C. for 15 minutes and allowed to cool before processing. Immunohistochemistry was performed for the detection of BrdU with a monoclonal primary antibody (Cell Signaling Technology #5292S, 1:5000), followed by a biotinylated secondary antibody (Jackson Immuno Research #715066151, 1:400). Immunostaining was revealed using Vector Labs VECTASTAIN® ELITE® ABC (PK-6100) and IMMPACT® DAB (SK-4105) kits. In the striatum and cortex, the percent area containing BrdU positive cells was assessed in wide-field images captured with a 4× objective using a Keyence BZ-X800 as described above. In the dentate gyrus, individual BrdU+ cells were counted by a treatment-blinded observer in 3 sections per rat (n=6/group). In the SVZ, BrdU+ staining was assessed by a treatment-blinded observer using NEUROLUCIDA® software (Microbrightfield) to map positive cells on the ischemic side (n=4-6).

Statistical analyses. Each experiment was performed in triplicate and then each repeated two or more times. The data for all experiments were averaged and plotted as means±SEM. Continuous data (MTD, EBST, westerns, and immunohistochemistry) was analyzed by 1- or 2-way Analysis of Variance (ANOVA) using GraphPad Prism v9.0. Multiple comparisons were performed with T-tests (2 groups) or Dunnet's tests (>2 groups). For ordinate data (blood clotting, Bederson, NDS), nonparametric tests were used (Kruskal-Wallis followed by Dunn's test). Significance was set at P<0.05.

### Example 2

### Sigma Receptor Binding by S1R Agonists

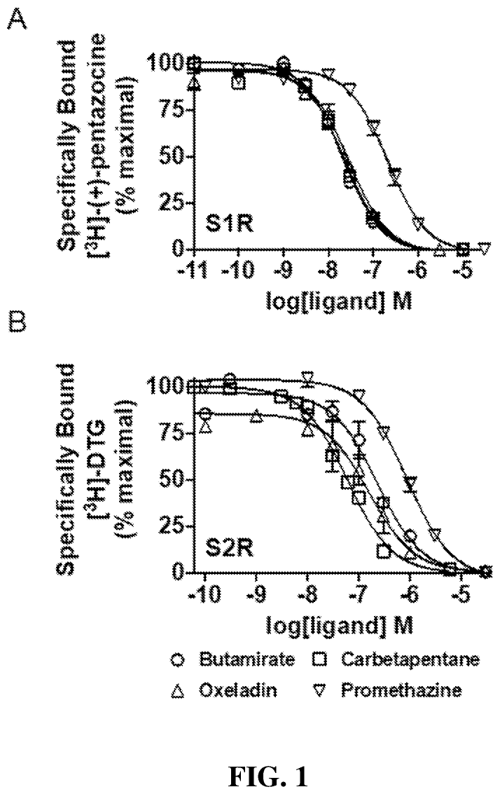

Referring to FIG. 1, the selectivity of potential S1R ligands over S2R was empirically evaluated. Human MCF-7 cells were used as the source of the S2R because these cells express the endogenous S2R (Vilner, John, et al., 1995; Wu & Bowen, 2008) but lack detectable levels of the S1R, allowing unambiguous detection of S2R activity in untransfected cells (Schetz, et al., 2007). MCF-7 cells stably expressing the cloned human S1R were used as the source of S1R. [³H]-DTG was used as the radioligand to probe for the S2R, and the high affinity S1R selective radioligand [³H]-(+)-pentazocine was used to probe for the S1R (Schetz, et al., 2007).

Activation of the S1R has been linked to antitussive effects, and antitussive agents like oxeladin, carbetapentane, and butamirate belong to the same structural class. These antitussive agents all had nanomolar affinities for both S1R (FIG. 1A) and S2R (FIG. 1B). Affinity values are listed in Table 1. Values are averages of 2-3 experiments±SEM, and each experiment was performed in triplicate.

TABLE 1
Effect of specific substructural modifications on selectivity for
S1R over S2R.

| | | S1R | S2R | S2B/S1R |
|---|---|---|---|---|
| | Substructozał | affinity | affinity (K [symbol] ) | ration |
| Compound | feature | (Kᵢ-nM) | nM) | (K [symbol] _{S2R}/K [symbol] ) |
| Carbetapentane | 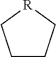 | 19.8 ± 2.3 | 55.7 ± 3.8 | 3 |
| Oxeladin | 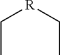 | 25 ± 4.3 | 148 ± 64 | 6 |
| Butamirate | 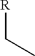 | 17.2 ± 1.7 | 214 ± 4 [symbol] | 10 |

[symbol]

indicates data missing or illegible when filed

Carbetapentane was 3-fold selective for S1R over the S2R, and oxeladin and butamirate were 9- and 10-fold selective for S1R over S2R, respectively. A pseudo-Hill slope equal to unity was the preferred model for all three antitussive agents at the S1R and S2R (P>0.05).

The antihistamine promethazine was previously found to bind S1R and was also included as a potential candidate drug. Promethazine was 5-fold selective for S1R over S2R with an affinity of 157 nM (FIG. 1).

### Example 3

### S1R Agonist-Mediated BDNF Release

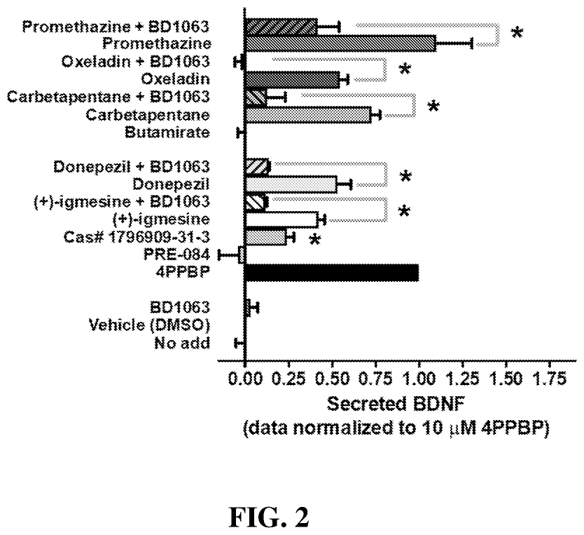

Referring to FIG. 2, functional selectivity of the potential S1R agonists was tested with an assay of BDNF release, as the prototypical S1R agonist, SA4503, and a novel S1R agonist, LS-1-137, stimulate BDNF secretion from neuronal and glial cells, respectively (Fujimoto, et al., 2012; Malik, et al., 2015). The MN9D neuronal cell line was selected because MN9D cells were found to produce a more robust BDNF release in response to SA4503 compared to the B104 neuronal cell line typically used for this assay (data not shown). In addition to producing robust response, the MN9D neuronal cell line also expresses BDNF (data not shown), S1R (Bₘₐₓ=1.5±0.26 pmol/mg membrane protein), and releases BDNF in response to treatment with the S1R agonist 4-PPBP (FIG. 2). Further, BDNF secreted from MN9D cells can be detected 24 hours after treatment with S1R ligand, whereas BDNF secreted from the B104 cell line takes 7 days to be detected (Fujimoto, et al., 2012).

An in situ ELISA approach was utilized because of its improved sensitivity related to its rapid capture of secreted BDNF (Balkowiec & Katz, 2000; Dalwadi, et al., 2017). All compounds were tested at an equimolar concentration of 10 μM, which represents maximum receptor occupancy (FIG. 1). Known S1R agonist 4-PPBP was used as the reference agonist and represented the maximum BDNF secretion response; all compounds were normalized to the 4-PPBP response (FIG. 2). Known reference compounds with BDNF-secreting activity also included (+)-igmesine, donepezil, and 2-Butoxyethyl 2-(diethylamino)-2-phenylacetate (Cas #1796909-31-3). The amount of BDNF secreted ranged from 24 to 103% of the maximum response (FIG. 2). Another known reference agonist, PRE-084, failed to stimulate BDNF secretion. Values are averages of 2-10 experiments±SEM, and each experiment was performed in triplicate. Statistical significance at P<0.05 was determined by ANOVA followed by a Bonferroni's post-hoc test and are marked with an asterisk. Efficacy of S1R ligands tested can be found in Table 2.

TABLE 2
Efficacy of S1R ligands that function as agonists of BDNF secretion.

| Compound | Efficacy of BDNF secretion |
|---|---|
| 4-PPBP | 1 |
| Promethazine | 1.09 ± 0.34 |
| Cas# 1796909-31-3 | 0.24 ± 0.04* |
| (+)-igmesine | 0.42 ± 0.03* |
| Donepezil | 0.53 ± 0.08* |
| Carbetapentane | 0.73 ± 0.05* |
| Oxeladin | 0.55 ± 0.04* |

Values are normalized to the maximal response of 10 μM 4-PPBP. Effcacies significantly different from 4-PPBP are marked with an asterisk (P < 0.05, one-way ANOVA, Bonferroni's post-hoc). All other efficacies were not significantly different. Values represent mean ± SEM.

With the exception of butamirate, all compounds were able to stimulate BDNF release, and their efficacy ranged from 54% to 144% (FIG. 2). BDNF secretion in response to treatment with the S1R agonists could also be reversed at a selective concentration of the known S1R antagonist BD1063 (15 nM) (FIG. 2).

### Example 4

### Determination of Maximum Tolerated Dose of S1R Agonist Oxeladin

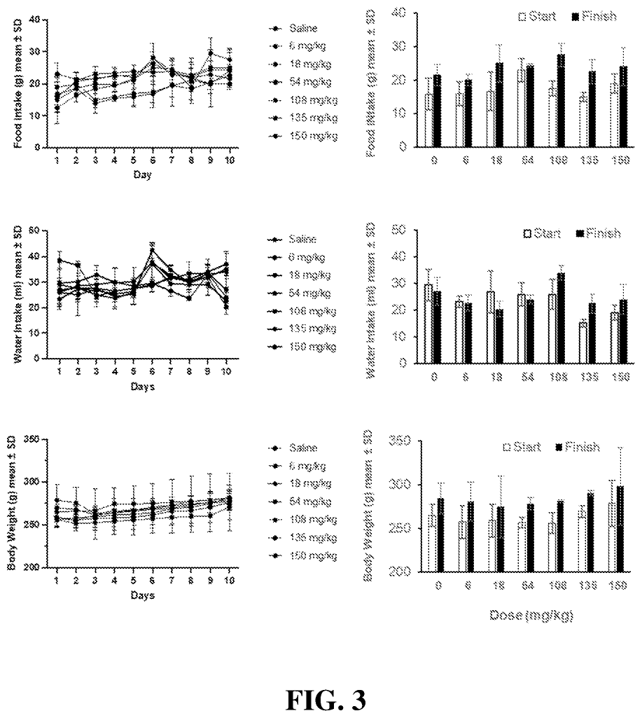

Referring to FIG. 3, there was a significant effect of time on weight (F_{1.61, 14.85}=23.71; P=0.002), food intake (F_{3,024, 42.34}=14.65; P<0.0001), and water intake (F_{2516, 35.22}=9.603; P=0.0002) over the course of dosing among male Sprague-Dawley rats groups receiving 6-150 mg/kg oxeladin and those receiving a saline control, but there were no significant differences among treatment groups in weight, food intake, or water intake over 10 days of daily oral gavage from 6 mg/kg to 150 mg/kg. There was no mortality or overt toxicity observed. These results indicate that, in some aspects, oxeladin does not affect ingestive behavior or weight, and a dose of at least up to 150 mg/kg oxeladin is well-tolerated and can be used in further experiments.

### Example 5

### Pharmacokinetics of Per Oral S1R Agonist Oxeladin

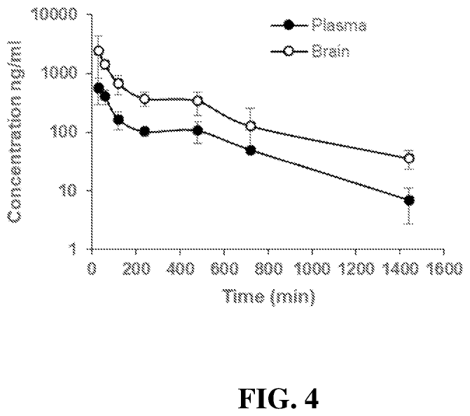

Referring to FIG. 4, brain pharmacokinetics for 135 mg/kg oxeladin administered to rats were as shown below in Table 3.

TABLE 3
Brain Pharmacokinetics of Oxeladin

| Parameter | Value |
|---|---|
| Terminal T_{1/2} | 319 min |
| (terminal elimination half-life) | |
| Cₘₐₓ | 2406 ng/g |
| (maximum concentration) | |
| (maximum concentration) | |
| Tₘₐₓ | 30 min |
| (time to obtain maximum concentration) | |
| AUCₗₐₛₜ | 423726 min*ng/g |
| (area under concentration-time curve to last | |
| measureable concentration) | |
| Blood-brain barrier Penetration | 3.5:1 |
| (C_{b}:Cₚ)* | |

*A molecule is deemed to be “brain penetrant” and to cross the blood-brain barrier if its brain-to-plasma concentration ratio (C_{b}:Cₚ) is >0.04.

Referring still to FIG. 4, plasma pharmacokinetics for 135 mg/kg oxeladin administered to rats were as shown below in Table 4.

TABLE 4
Plasma Pharmacokinetics of Oxeladin

| Parameter | Value |
|---|---|
| Terminal T_{1/2} | 249 min |
| (terminal elimination half-life) | |
| Cₘₐₓ | 5666 ng/g |
| (maximum concentration) | |
| Tₘₐₓ | 30 min |
| (time to obtain maximum concentration) | |
| AUCₗₐₛₜ | 122293 min*ng/ml |
| (area under concentration-time curve to last | |
| measureable concentration) | |
| Vz/F | 387.8 ml |
| (apparent volume of distribution) | |
| CL/F | 1.08 ml/min |
| (apparent oral clearance) | |

Despite a LogP of 4.43, oxeladin was detected in the plasma and brain 30 minutes after dosing. The terminal half-life in plasma was calculated as 249 min, while that in brain was 319 min, suggesting that brain concentrations reflect peripheral absorption. These experiments demonstrate the ability of the S1R agonist oxeladin, which exhibited a C_{b}:Cₚ of 3.5:1, to cross the blood-brain barrier after oral administration. These results indicate that, in some aspects, oxeladin displayed favorable pharmacokinetics in plasma and brain after oral dosing.

### Example 6

### Effect of Oral Oxeladin Administration on Thrombosis

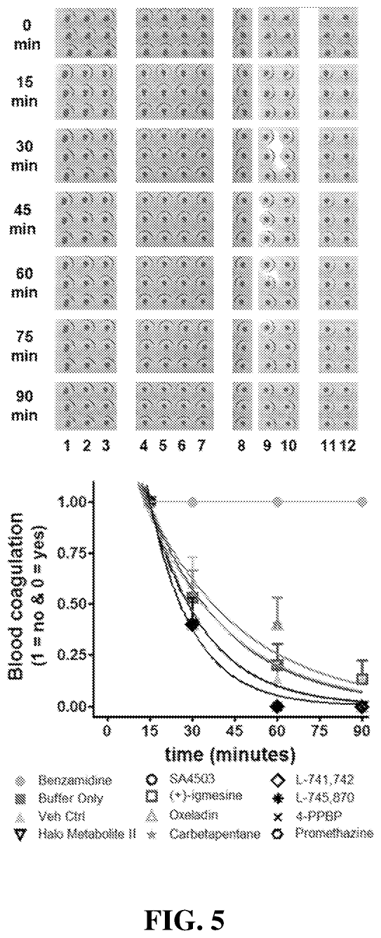

Referring to FIG. 5, the effect on thrombotic activity of test drugs, benzamidine anticoagulant positive control, or untreated or vehicle negative controls on whole blood from donor rats was assessed by tilting treated blood samples at a 450 angle to observe coagulation (i.e., no streaking) between 0 and 90 minutes (top panel). Blood coagulation was represented by a value between 1 (no streaking, coagulation) and 0 (streaking, less or no coagulation), and values were assigned for each test drug, benzamidine, and untreated and vehicle controls (bottom panel). Quantitative results from these clotting assays, including coagulation half-life, are summarized below in Table 5.

TABLE 5
Thrombotic Activity of Sigma 1 Compounds

| | | Concentration | Coagulation |
|---|---|---|---|
| | | Tested | Half-Life |
| Lane | Compound | (μM) | (λ_{1/2}, minutes ± SD) |
| 1 | Benzamidine | 3000 | >1,440 |
| | (positive control) | | |
| 2 | Buffer Only | N/A | 13 ± 7 |
| | (negative control) | | |
| 3 | Vehicle Control (1:1000 | N/A | 19 ± 14 |
| | v/v DMSO) | | |
| 4 | Haloperidol Metabolite II | 100 | 14 ± 11 |
| 5 | SA4503 | 30 | 11 ± 6 |
| 6 | (+)-igmesine | 100 | 19 ± 16 |
| 7 | Oxeladin | 1000 | 11 ± 6 |
| 8 | L-741, 742 | 10 | 11 ± 6 |
| 9 | L-745, 870 | 50 | 11 ± 6 |
| 10 | Carbetapentane | 1000 | 23 ± 18 |
| 11 | 4-PPBP | 100 | 11 ± 6 |
| 12 | Promethazine | 1000 | 11 ± 6 |

The coagulation half-life of oxeladin and promethazine was 11±6 for both compounds. By way of comparison, the coagulation half-life of the negative and vehicle controls was 13±7 and 19±14, whereas the coagulation half-life of the positive control was >1,440. These experiments demonstrate that, in some aspects, S1R agonists oxeladin and promethazine do not alter thrombosis in blood after oral administration compared to negative and vehicle controls.

### Example 7

### Effect of Oral Oxeladin Administration on Neurological Function

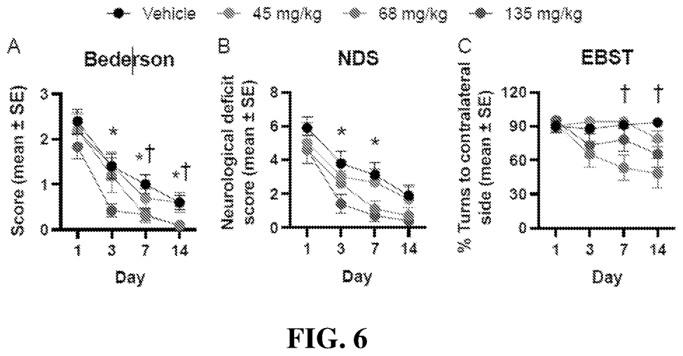

A treatment-blinded observer conducted behavioral assessments on days 3, 7, and 14 after tMCAO. Referring to FIG. 6, a treatment-blinded observer assessed the effects of administration of 45 mg/kg, 68 mg/kg, or 135 mg/kg oxeladin dose on neurological function in rats having neurological deficits on days 3, 7, and 14 after middle cerebral artery occlusion.

The observer scored the rats using the Bederson test, which evaluates global neurological function and grades rodents on a scale of 0-5. Grade 0 (no defects) is assigned if animals extend both forelimbs toward the floor when suspended above the floor. Grade 1 (mild defects) is given when animals show forelimb flexion without any other abnormality. Grade 2 (modest defects) is given when animals display forelimb flexion and decreased resistance to lateral push toward the paretic side. Animals further showing circling behavior are scored Grade 3 (severe defects). As shown in FIG. 6 (top panel), all treatment groups displayed similar behavioral deficits 24 hours after tMCAO. Administration of both 68 mg/kg and 135 mg/kg oxeladin significantly improved neurological function on days 7 and 14, compared to vehicle. Dunn's multiple comparison tests for each day demonstrated significant improvements with 135 mg/kg on days 3, 7, and 14 (P=0.04).

The observer also scored the rats using an 11-point neurological deficit score (NDS). As shown in FIG. 6 (middle panel), administration of the 135 mg/kg oxeladin dose significantly improved neurological function on days 3 and 7, compared to vehicle. The NDS (Kruskal-Wallis, P=0.029) revealed significant improvements in animals receiving 135 mg/kg oxeladin on day 7 (Dunn's P=0.028) and a trend for improvement on day 3 (Dunn's, P=0.072).

The observer also scored the rats using the Elevated Body Swing Test (EBST) in which rats lifted by the tail and held vertically swing their heads to the left or to the right. Whereas healthy animals on a group level are likely to swing approximately 50% to either side, animals with a unilateral cerebral lesion, e.g. ischemic stroke, should present with a dominant/biased swing direction. As shown in FIG. 6 (bottom panel), administration of the 68 mg/kg oxeladin dose significantly improved function on days 7 and 14, compared to vehicle. On the elevated body swing test (EBST), rats receiving 68 mg/kg, but not 135 mg/kg showed improvement compared to vehicle on days 7 (Dunn's P=0.044) and 14 (Dunn's, P=0.016).

Together, these results indicate that, in some aspects, oxeladin improves functional recovery after transient middle cerebral artery occlusion.

### Example 8

### Infarct Size and Inflammation

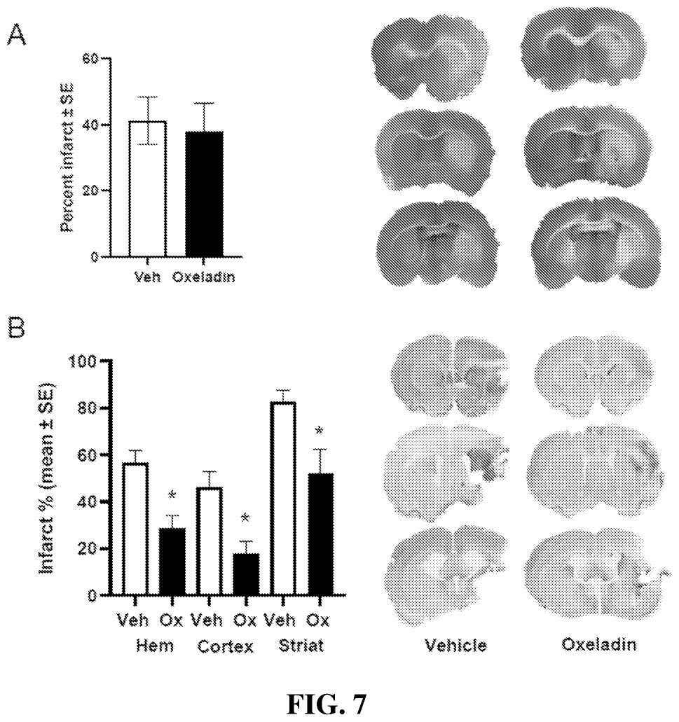

No significant difference in infarct size with rapid TTC staining was observed 24 hours after a single dose of oxeladin (FIG. 7A). In contrast, 14 days after tMCAO, infarct size assessed with Nissl staining did reveal group differences (FIG. 7B). Oxeladin significantly reduced infarct extent in the whole hemisphere, cortex, and striatum (t-test P<0.05).). These results demonstrate that, in some aspects, oxeladin reduces delayed infarct after tMCAO.

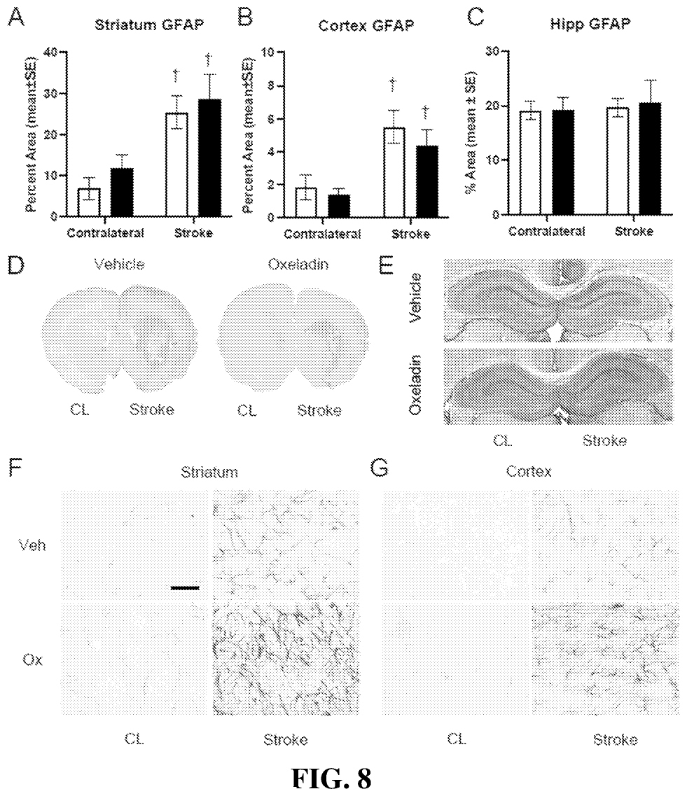

Extensive gliosis revealed with GFAP was observed in the ischemic hemisphere 14 days after tMCAO in both the vehicle and oxeladin-treated groups (FIG. 8). A significant effect of stroke was observed in both the striatum (F_{1,12}=27.56, P=0.002, FIG. 8A) and the cerebral cortex (F_{1,14}=23.17, P=0.003, FIG. 8B), but not in the dorsal hippocampus (FIG. 8C). However, there were no differences between vehicle and oxeladin treated groups. These results demonstrate that, in some aspects, oxeladin does not alter astrogliosis after tMCAO.

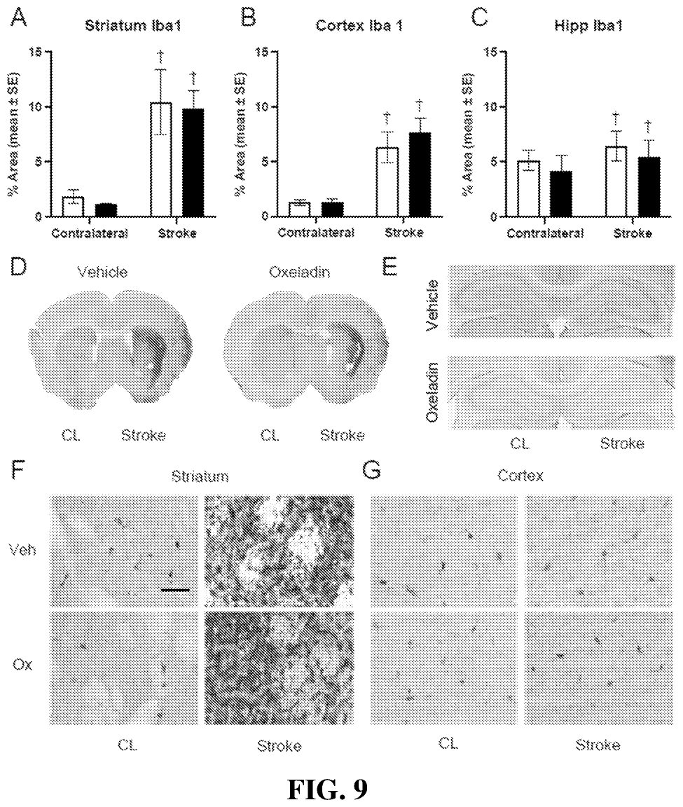

Similarly, extensive microgliosis on in the ischemic hemisphere was revealed with Iba1 staining (FIG. 9). A significant effect of tMCAO was observed in the striatum (F_{1,6}=19.8, P=0.004, FIG. 9A), cerebral cortex (F_{1,6}=7.154, P=0.037, FIG. 9B) and dorsal hippocampus (F_{1,6}=16.20, P=0.007, FIG. 9C). However, there were no differences observed between the vehicle and oxeladin treated groups. These results demonstrate that, in some aspects, oxeladin does not alter microgliosis after tMCAO.

### Example 9

### BDNF Levels

BDNF expression is highly correlated with stroke recovery, and an association between pharmaceutical treatments for cerebral ischemia and BDNF levels in the brain has been demonstrated. Unfortunately, however, peptides such as BDNF do not possess favorable pharmacological properties for peripheral administration.

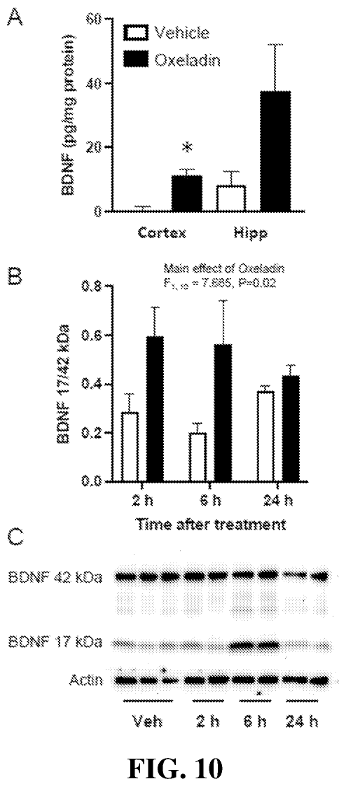

Ten days of treatment with 135 mg/kg of oxeladin resulted in a significant increase in BDNF levels in the cerebral cortex (t-test P<0.05) but not in the hippocampus when measured by ELISA (FIG. 10A). A single dose of 135 mg/kg oxeladin administered 48 hours after tMCAO significantly increased (F_{1,10}=7.685, P=0.02) the ratio of mature 17 kDa BDNF compared to 42 kDa pro-BDNF in the ischemic penumbra (FIGS. 10B, 10C). These experiments suggest that, in some aspects, oxeladin increases BDNF levels in the brain and that BDNF release plays an important role in the restorative actions of oxeladin.

### Example 10

### Proliferation in the Brain

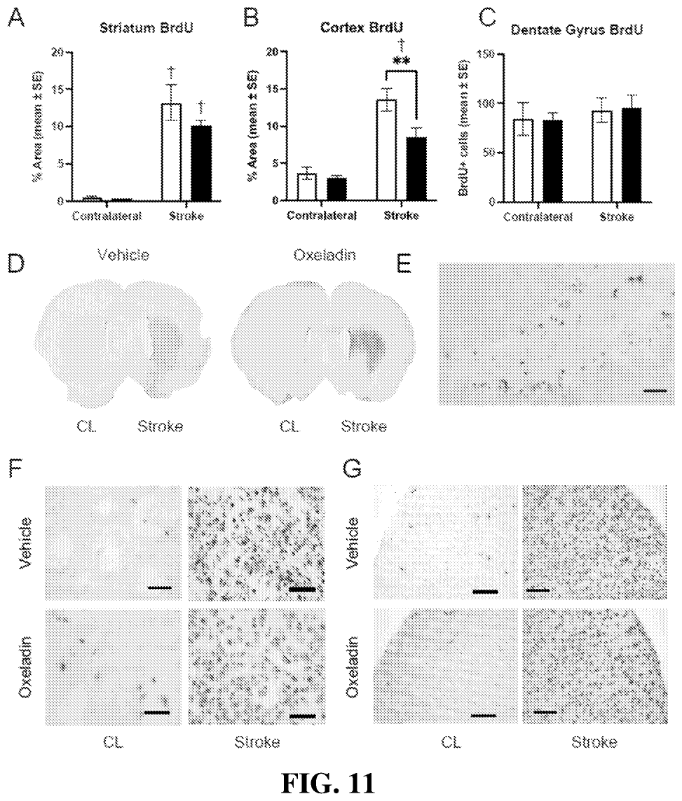

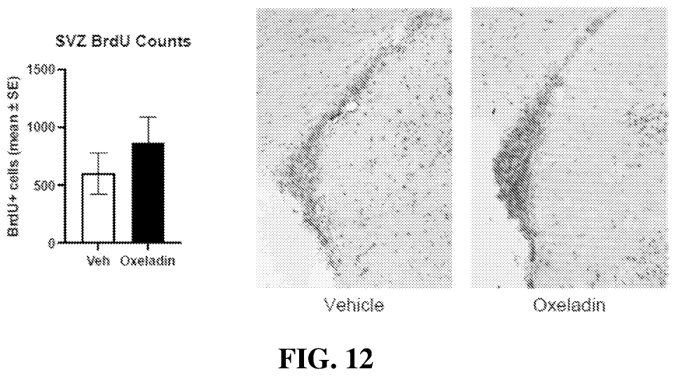

Fourteen days after tMCAO BrdU positive cells were quantified in rats treated with vehicle or 135 mg/kg oxeladin. A significantly larger area of the striatum (F_{1,10}=73.54, P<0.0001, FIG. 11A) and cortex (F_{1,10}=35.85, P=0.0001, FIG. 11B) in the ischemic hemisphere contained BrdU+ cells compared to the contralateral hemisphere, but no differences were observed in BrdU+ cells in the dentate gyrus (FIG. 11C). In the cerebral cortex, a significantly smaller area of BrdU+ cells appeared in the oxeladin-treated rats compared to vehicle controls (Treatment effect F_{1,10}=13.93, P=0.004; Bonferroni's multiple comparisons test P=0.007, FIG. 11B). In the subventricular zone (SVZ) on the ischemic side of the brain, there were no differences in BrdU+ cells between treatment groups (FIG. 12). These results suggest that, in some aspects, oxeladin does not enhance cell proliferation in the brain or SVZ after tMCAO. Failure to observe significant differences in endogenous stem cell proliferation in oxeladin-treated rats raises the potential that BDNF increases (FIG. 10) may, in some aspects, improve function of existing cells rather than promote an increase in neuronal cells.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

### REFERENCES

The following references and those cited elsewhere herein, to the extent that they provide exemplary procedural or other details supplementary to those set forth herein, are specifically incorporated herein by reference.
1. Al-Nabulsi, I., Mach, R. H., Wang, L. M., Wallen, C. A., Keng, P. C., Sten, K., Childers, S. R., & Wheeler, K. T. (1999). Effect of ploidy, recruitment, environmental factors, and tamoxifen treatment on the expression of sigma-2 receptors in proliferating and quiescent tumour cells. British journal of cancer, 81, 925-933.
2. Allahtavakoli, M., & Jarrott, B. (2011). Sigma-1 receptor ligand PRE-084 reduced infarct volume, neurological deficits, pro-inflammatory cytokines and enhanced anti-inflammatory cytokines after embolic stroke in rats. Brain Res Bull, 85, 219-224.
3. Alonso, G., Phan, V., Guillemain, I., Saunier, M., Legrand, A., Anoal, M., & Maurice, T. (2000). Immunocytochemical localization of the sigma(1) receptor in the adult rat central nervous system. Neuroscience, 97, 155-170.
4. Balkowiec, A., & Katz, D. M. (2000). Activity-dependent release of endogenous brain-derived neurotrophic factor from primary sensory neurons detected by ELISA in situ. J Neurosci, 20, 7417-7423.
5. Bederson, J. B., Pitts, L. H., Tsuji, M., Nishimura, M. C., Davis, R. T., Bartkowski, H. (1986). Rat middle cerebral artery occlusion: evaluation of the model and development of a neurologic examination. Stroke, 17, 472-476.
6. Borlongan, C. V., Sanberg, P. R. (1995). Elevated body swing test: a new behavioral parameter for rats with 6-hydroxydopamine-induced hemiparkinsonism. J Neurosci, 15, 5372-5378.
7. Bowen, W. D. (2000). Sigma receptors: recent advances and new clinical potentials. Pharm Acta Helv, 74, 211-218.
8. Brown, C., Fezoui, M., Selig, W. M., Schwartz, C. E., & Ellis, J. L. (2004). Antitussive activity of sigma-1 receptor agonists in the guinea-pig. Br J Pharmacol, 141, 233-240.
9. Bruna, J., Videla, S., Argyriou, A. A., Velasco, R., Villoria, J., Santos, C., Nadal, C., Cavaletti, G., Alberti, P., Briani, C., Kalofonos, H. P., Cortinovis, D., Sust, M., Vaque, A., Klein, T., & Plata-Salaman, C. (2018). Efficacy of a Novel Sigma-1 Receptor Antagonist for Oxaliplatin-Induced Neuropathy: A Randomized, Double-Blind, Placebo-Controlled Phase IIa Clinical Trial. Neurotherapeutics, 15, 178-189.
10. Cunha, C., Brambilla, R., & Thomas, K. L. (2010). A simple role for BDNF in learning and memory? Front Mol Neurosci, 3, 1.
11. Dalwadi, D. A., Kim, S., Schetz, J., Schreihofer, D. A., Kim, S. (2022). Brain-derived neurotrophic factor for high-throughput evaluation of selective Sigma-1 receptor ligands. J Pharmacol Toxicol Methods, 113, 107129.
12. Dalwadi, D. A., Kim, S., & Schetz, J. A. (2017). Activation of the sigma-1 receptor by haloperidol metabolites facilitates brain-derived neurotrophic factor secretion from human astroglia. Neurochem Int, 105, 21-31.
13. Fu, Y., Li, J., & Lee, A. S. (2007). GRP78/BiP inhibits endoplasmic reticulum BIK and protects human breast cancer cells against estrogen starvation-induced apoptosis. Cancer Res, 67, 3734-3740.
14. Fujimoto, M., Hayashi, T., Urfer, R., Mita, S., & Su, T. P. (2012). Sigma-1 receptor chaperones regulate the secretion of brain-derived neurotrophic factor. Synapse, 66, 630-639.
15. Garces-Ramirez, L., Green, J. L., Hiranita, T., Kopajtic, T. A., Mereu, M., Thomas, A. M., Mesangeau, C., Narayanan, S., McCurdy, C. R., Katz, J. L., & Tanda, G. (2011). Sigma receptor agonists: receptor binding and effects on mesolimbic dopamine neurotransmission assessed by microdialysis. Biol Psychiatry, 69, 208-217.
16. Gekker, G., Hu, S., Sheng, W. S., Rock, R. B., Lokensgard, J. R., & Peterson, P. K. (2006). Cocaine-induced HIV-1 expression in microglia involves sigma-1 receptors and transforming growth factor-betal. Int Immunopharmacol, 6, 1029-1033.
17. Guitart, X., Codony, X., & Monroy, X. (2004). Sigma receptors: biology and therapeutic potential. Psychopharmacology (Berl), 174, 301-319.
18. Hashimoto, K., Scheffel, U., & London, E. D. (1995). In vivo labeling of sigma receptors in mouse brain with [³H]4-phenyl-1-(4-phenylbutyl)piperidine. Synapse, 20, 85-90.
19. Hayashi, T., Maurice, T., & Su, T. P. (2000). Ca(2+) signaling via sigma(1)-receptors: novel regulatory mechanism affecting intracellular Ca(2+) concentration. J Pharmacol Exp Ther, 293, 788-798.
20. Hayashi, T., & Su, T. P. (2004). Sigma-1 receptors at galactosylceramide-enriched lipid microdomains regulate oligodendrocyte differentiation. Proc Natl Acad Sci USA, 101, 14949-14954.
21. Hayashi, T., & Su, T. P. (2007). Sigma-1 receptor chaperones at the ER-mitochondrion interface regulate Ca(2+) signaling and cell survival. Cell, 131, 596-610.
22. Hempstead, B. L., Rabin, S. J., Kaplan, L., Reid, S., Parada, L. F., & Kaplan, D. R. (1992). Overexpression of the trk tyrosine kinase rapidly accelerates nerve growth factor-induced differentiation. Neuron, 9, 883-896.
23. Hong, W., Nuwayhid, S. J., & Werling, L. L. (2004). Modulation of bradykinin-induced calcium changes in SH-SY5Y cells by neurosteroids and sigma receptor ligands via a shared mechanism. Synapse, 54, 102-110.
24. Hornick, J. R., Vangveravong, S., Spitzer, D., Abate, C., Berardi, F., Goedegebuure, P., Mach, R. H., & Hawkins, W. G. (2012). Lysosomal membrane permeabilization is an early event in Sigma-2 receptor ligand mediated cell death in pancreatic cancer. J Exp Clin Cancer Res, 31, 41.
25. Huang, Y. S., Lu, H. L., Zhang, L. J., & Wu, Z. (2014). Sigma-2 receptor ligands and their perspectives in cancer diagnosis and therapy. Med Res Rev, 34, 532-566.
26. Iniguez, M. A., Punzon, C., Nieto, R., Burgueno, J., Vela, J. M., & Fresno, M. (2013). Inhibitory effects of sigma-2 receptor agonists on T lymphocyte activation. Front Pharmacol, 4, 23.
27. Ishima, T., Fujita, Y., & Hashimoto, K. (2014). Interaction of new antidepressants with sigma-1 receptor chaperones and their potentiation of neurite outgrowth in PC12 cells. Eur J Pharmacol, 727, 167-173.
28. Ishima, T., & Hashimoto, K. (2012). Potentiation of nerve growth factor-induced neurite outgrowth in PC12 cells by ifenprodil: the role of sigma-1 and IP3 receptors. PLoS One, 7, e37989.
29. Ishima, T., Nishimura, T., Iyo, M., & Hashimoto, K. (2008). Potentiation of nerve growth factor-induced neurite outgrowth in PC12 cells by donepezil: role of sigma-1 receptors and IP3 receptors. Prog Neuropsychopharmacol Biol Psychiatry, 32, 1656-1659.
30. Jiang, G., Mysona, B., Dun, Y., Gnana-Prakasam, J. P., Pabla, N., Li, W., Dong, Z., Ganapathy, V., & Smith, S. B. (2006). Expression, subcellular localization, and regulation of sigma receptor in retinal muller cells. Investigative ophthalmology & visual science, 47, 5576-5582.
31. Jin, J. L., Fang, M., Zhao, Y. X., & Liu, X. Y. (2015). Roles of sigma-1 receptors in Alzheimer's disease. Int J Clin Exp Med, 8, 4808-4820.
32. Kikuchi-Utsumi, K., & Nakaki, T. (2008). Chronic treatment with a selective ligand for the sigma-1 receptor chaperone, SA4503, up-regulates BDNF protein levels in the rat hippocampus. Neurosci Lett, 440, 19-22.
33. Kim, S., Radhakrishnan, U. P., Rajpurohit, S. K., Kulkarni, V., Jagadeeswaran, P. (2010). Vivo-Morpholino knockdown of alphaIIb: A novel approach to inhibit thrombocyte function in adult zebrafish. Blood Cells Mol Dis, 44, 169-174.
34. Klette, K. L., DeCoster, M. A., Moreton, J. E., & Tortella, F. C. (1995). Role of calcium in sigma-mediated neuroprotection in rat primary cortical neurons. Brain Res, 704, 31-41.
35. Korte, M., Carroll, P., Wolf, E., Brem, G., Thoenen, H., & Bonhoeffer, T. (1995). Hippocampal long-term potentiation is impaired in mice lacking brain-derived neurotrophic factor. Proc Natl Acad Sci USA, 92, 8856-8860.
36. Kourrich, S., Su, T. P., Fujimoto, M., & Bonci, A. (2012). The sigma-1 receptor: roles in neuronal plasticity and disease. Trends Neurosci, 35, 762-771.
37. Lee, I. T., Chen, S., & Schetz, J. A. (2008). An unambiguous assay for the cloned human sigmal receptor reveals high affinity interactions with dopamine D4 receptor selective compounds and a distinct structure-affinity relationship for butyrophenones. Eur J Pharmacol, 578, 123-136.
38. Lewin, G. R., & Barde, Y. A. (1996). Physiology of the neurotrophins. Annu Rev Neurosci, 19, 289-317.
39. Luedtke, R. R., Perez, E., Yang, S. H., Liu, R., Vangveravong, S., Tu, Z., Mach, R. H., & Simpkins, J. W. (2012). Neuroprotective effects of high affinity Sigmal receptor selective compounds. Brain Res, 1441, 17-26.
40. Malik, M., Rangel-Barajas, C., Sumien, N., Su, C., Singh, M., Chen, Z., Huang, R. Q., Meunier, J., Maurice, T., Mach, R. H., & Luedtke, R. R. (2015). The effects of sigma (sigmal) receptor-selective ligands on muscarinic receptor antagonist-induced cognitive deficits in mice. Br J Pharmacol, 172, 2519-2531.
41. Marrazzo, A., Caraci, F., Salinaro, E. T., Su, T. P., Copani, A., & Ronsisvalle, G. (2005). Neuroprotective effects of sigma-1 receptor agonists against beta-amyloid-induced toxicity. Neuroreport, 16, 1223-1226.
42. Maurice, T., Su, T. P., & Privat, A. (1998). Sigmal (Sigma-1) receptor agonists and neurosteroids attenuate B25-35-amyloid peptide-induced amnesia in mice through a common mechanism. Neuroscience, 83, 413-428.
43. Mendes, C. C., Gomes, D. A., Thompson, M., Souto, N. C., Goes, T. S., Goes, A. M., Rodrigues, M. A., Gomez, M. V., Nathanson, M. H., & Leite, M. F. (2005). The type III inositol 1,4,5-trisphosphate receptor preferentially transmits apoptotic Ca2+ signals into mitochondria. J Biol Chem, 280, 40892-40900.
44. Monassier, L., Manoury, B., Bellocq, C., Weissenburger, J., Greney, H., Zimmermann, D., Ehrhardt, J. D., Jaillon, P., Baro, I., & Bousquet, P. (2007). sigma(2)-receptor ligand-mediated inhibition of inwardly rectifying K(+) channels in the heart. J Pharmacol Exp Ther, 322, 341-350.
45. Niitsu, T., Fujisaki, M., Shiina, A., Yoshida, T., Hasegawa, T., Kanahara, N., Hashimoto, T., Shiraishi, T., Fukami, G., Nakazato, M., Shirayama, Y., Hashimoto, K., & Iyo, M. (2012). A randomized, double-blind, placebo-controlled trial of fluvoxamine in patients with schizophrenia: a preliminary study. J Clin Psychopharmacol, 32, 593-601.
46. Nishimura, T., Ishima, T., Iyo, M., & Hashimoto, K. (2008). Potentiation of nerve growth factor-induced neurite outgrowth by fluvoxamine: role of sigma-1 receptors, IP3 receptors and cellular signaling pathways. PLoS One, 3, e2558.
47. Okuyama, S., & Nakazato, A. (1996). NE-100: A novel sigma receptor antagonist. CNS Drug Reviews, 2, 226-237.
48. Oppong-Gyebi, A., Metzger, D., Doan, T., Han, J., Vann, P. H., Yockey, R. A., Sumien, N., Schreihofer, D. A. (2022). Long-term hypogonadism diminishes the neuroprotective effects of dietary genistein in young adult ovariectomized rats after transient focal ischemia. J Neurosci Res, 100, 598-619.
49. Ostenfeld, M. S., Fehrenbacher, N., Hoyer-Hansen, M., Thomsen, C., Farkas, T., & Jaattela, M. (2005). Effective tumor cell death by sigma-2 receptor ligand siramesine involves lysosomal leakage and oxidative stress. Cancer Res, 65, 8975-8983.
50. Palacios, G., Muro, A., Vela, J. M., Molina-Holgado, E., Guitart, X., Ovalle, S., & Zamanillo, D. (2003). Immunohistochemical localization of the sigmal-receptor in oligodendrocytes in the rat central nervous system. Brain Res, 961, 92-99.
51. Palacios, G., Muro, A., Verdu, E., Pumarola, M., & Vela, J. M. (2004). Immunohistochemical localization of the sigmal receptor in Schwann cells of rat sciatic nerve. Brain Res, 1007, 65-70.
52. Peviani, M., Salvaneschi, E., Bontempi, L., Petese, A., Manzo, A., Rossi, D., Salmona, M., Collina, S., Bigini, P., & Curti, D. (2014). Neuroprotective effects of the Sigma-1 receptor (S1R) agonist PRE-084, in a mouse model of motor neuron disease not linked to SOD1 mutation. Neurobiol Dis, 62, 218-232.
53. Robson, M. J., Turner, R. C., Naser, Z. J., McCurdy, C. R., O'Callaghan, J. P., Huber, J. D., & Matsumoto, R. R. (2014). SN79, a sigma receptor antagonist, attenuates methamphetamine-induced astrogliosis through a blockade of OSMR/gp130 signaling and STAT3 phosphorylation. Exp Neurol, 254, 180-189.
54. Rodriguez-Munoz, M., Onetti, Y., Cortes-Montero, E., Garzon, J., & Sanchez-Blazquez, P. (2018). Cannabidiol enhances morphine antinociception, diminishes NMDA-mediated seizures and reduces stroke damage via the Sigma-1 receptor. Mol Brain, 11, 51.
55. Rossi, D., Pedrali, A., Urbano, M., Gaggeri, R., Serra, M., Fernandez, L., Fernandez, M., Caballero, J., Ronsisvalle, S., Prezzavento, O., Schepmann, D., Wuensch, B., Peviani, M., Curti, D., Azzolina, O., & Collina, S. (2011). Identification of a potent and selective sigma(1) receptor agonist potentiating NGF-induced neurite outgrowth in PC12 cells. Bioorg Med Chem, 19, 6210-6224.
56. Ruscher, K., Inacio, A. R., Valind, K., Rowshan Ravan, A., Kuric, E., & Wieloch, T. (2012). Effects of the sigma-1 receptor agonist 1-(3,4-dimethoxyphenethyl)-4-(3-phenylpropyl)-piperazine dihydro-chloride on inflammation after stroke. PLoS One, 7, e45118.
57. Ruscher, K., Shamloo, M., Rickhag, M., Ladunga, I., Soriano, L., Gisselsson, L., Toresson, H., Ruslim-Litrus, L., Oksenberg, D., Urfer, R., Johansson, B. B., Nikolich, K., & Wieloch, T. (2011). The sigma-1 receptor enhances brain plasticity and functional recovery after experimental stroke. Brain: a journal of neurology, 134, 732-746.
58. Ryskamp, D. A., Korban, S., Zhemkov, V., Kraskovskaya, N., & Bezprozvanny, I. (2019). Neuronal Sigma-1 Receptors: Signaling Functions and Protective Roles in Neurodegenerative Diseases. Front Neurosci, 13, 862.
59. Sato, S., Kawamata, T., Kobayashi, T., & Okada, Y. (2014). Antidepressant fluvoxamine reduces cerebral infarct volume and ameliorates sensorimotor dysfunction in experimental stroke. Neuroreport, 25, 731-736.
60. Schetz, J. A., Perez, E., Liu, R., Chen, S., Lee, I., & Simpkins, J. W. (2007). A prototypical Sigma-1 receptor antagonist protects against brain ischemia. Brain Res, 1181, 1-9.
61. Schmitt, C. A., & Dirsch, V. M. (2009). Modulation of endothelial nitric oxide by plant-derived products. Nitric Oxide, 21, 77-91.
62. Schneider, L. S., Thomas, R. G., Hendrix, S., Rissman, R. A., Brewer, J. B., Salmon, D. P., Oltersdorf, T., Okuda, T., Feldman, H. H., & Alzheimer's Disease Cooperative Study, T. S. G. (2019). Safety and Efficacy of Edonerpic Maleate for Patients With Mild to Moderate Alzheimer Disease: A Phase 2 Randomized Clinical Trial. JAMA Neurol.
63. Searovic, P., Alonso, M., Oses, C., Pereira-Flores, K., Velarde, V., & Saez, C. G. (2009). Effect of tamoxifen and retinoic acid on bradykinin induced proliferation in MCF-7 cells. J Cell Biochem, 106, 473-481.
64. Shen, Y. C., Wang, Y. H., Chou, Y. C., Liou, K. T., Yen, J. C., Wang, W. Y., & Liao, J. F. (2008). Dimemorfan protects rats against ischemic stroke through activation of sigma-1 receptor-mediated mechanisms by decreasing glutamate accumulation. J Neurochem, 104, 558-572.
65. Su, T. P., Hayashi, T., Maurice, T., Buch, S., & Ruoho, A. E. (2010). The sigma-1 receptor chaperone as an inter-organelle signaling modulator. Trends Pharmacol Sci, 31, 557-566.
66. Swanson, R. A., Morton, M. T., Tsao-Wu, G., Savalos, R. A., Davidson, C., Sharp, F. R. (1990). A semiautomated method for measuring brain infarct volume. J Cereb Blood Flow Metab, 10, 290-293.
67. Takebayashi, M., Hayashi, T., & Su, T. P. (2002). Nerve growth factor-induced neurite sprouting in PC12 cells involves sigma-1 receptors: implications for antidepressants. J Pharmacol Exp Ther, 303, 1227-1237.
68. Urfer, R., Moebius, H. J., Skoloudik, D., Santamarina, E., Sato, W., Mita, S., Muir, K. W., & Cutamesine Stroke Recovery Study, G. (2014). Phase II trial of the Sigma-1 receptor agonist cutamesine (SA4503) for recovery enhancement after acute ischemic stroke. Stroke, 45, 3304-3310.
69. Villard, V., Espallergues, J., Keller, E., Alkam, T., Nitta, A., Yamada, K., Nabeshima, T., Vamvakides, A., & Maurice, T. (2009). Antiamnesic and neuroprotective effects of the aminotetrahydrofuran derivative ANAVEX1-41 against amyloid beta(25-35)-induced toxicity in mice. Neuropsychopharmacology, 34, 1552-1566.
70. Vilner, B. J., de Costa, B. R., & Bowen, W. D. (1995). Cytotoxic effects of sigma ligands: sigma receptor-mediated alterations in cellular morphology and viability. J Neurosci, 15, 117-134.
71. Vilner, B. J., John, C. S., & Bowen, W. D. (1995). Sigma-1 and sigma-2 receptors are expressed in a wide variety of human and rodent tumor cell lines. Cancer Res, 55, 408-413.
72. Whittemore, E. R., Ilyin, V. I., & Woodward, R. M. (1997). Antagonism of N-methyl-D-aspartate receptors by sigma site ligands: potency, subtype-selectivity and mechanisms of inhibition. J Pharmacol Exp Ther, 282, 326-338.
73. Wu, Z., & Bowen, W. D. (2008). Role of sigma-1 receptor C-terminal segment in inositol 1,4,5-trisphosphate receptor activation: constitutive enhancement of calcium signaling in MCF-7 tumor cells. J Biol Chem, 283, 28198-28215.
74. Zeng, C., Rothfuss, J., Zhang, J., Chu, W., Vangveravong, S., Tu, Z., Pan, F., Chang, K. C., Hotchkiss, R., & Mach, R. H. (2012). Sigma-2 ligands induce tumour cell death by multiple signalling pathways. British journal of cancer, 106, 693-701.
75. Zeng, C., Rothfuss, J. M., Zhang, J., Vangveravong, S., Chu, W., Li, S., Tu, Z., Xu, J., & Mach, R. H. (2014). Functional assays to define agonists and antagonists of the sigma-2 receptor. Anal Biochem, 448, 68-74.
76. Zeng, C., Vangveravong, S., McDunn, J. E., Hawkins, W. G., & Mach, R. H. (2013). Sigma-2 receptor ligand as a novel method for delivering a SMAC mimetic drug for treating ovarian cancer. British journal of cancer, 109, 2368-2377.
77. Zheng, P. (2009). Neuroactive steroid regulation of neurotransmitter release in the CNS: action, mechanism and possible significance. Prog Neurobiol, 89, 134-152.
78. Zou, L. B., Yamada, K., Sasa, M., Nakata, Y., & Nabeshima, T. (2000). Effects of sigma(1) receptor agonist SA4503 and neuroactive steroids on performance in a radial arm maze task in rats. Neuropharmacology, 39, 1617-1627.

1. A method of treating ischemic injury in a subject having an ischemic injury, the method comprising administering to the subject a composition comprising an effective amount of a Sigma receptor agonist to treat the ischemic injury in the subject, wherein the composition is administered at least about 6 hours after occurrence of the ischemic injury.

2. The method of claim 1, wherein the ischemic injury is induced by a cerebral ischemia.

3. The method of claim 2, wherein the cerebral ischemia is thrombotic ischemia, embolic ischemia, or hypoperfusion.

4. The method of claim 3, wherein the cerebral ischemia is ischemic stroke.

5. The method of claim 1, wherein the Sigma receptor agonist is a Sigma-1 receptor agonist.

6. The method of claim 1, wherein the Sigma receptor agonist comprises an antitussive, an antipsychotic, an antidepressant, a neurosteroid, a neuroleptic, or a psychostimulant, or any combination thereof.

7. The method of claim 1, wherein the Sigma receptor agonist comprises oxeladin.

8. The method of claim 1, wherein the Sigma receptor agonist comprises promethazine.

9. The method of claim 1, wherein the Sigma receptor agonist crosses the subject's blood-brain barrier.

10. The method of claim 1, wherein the ischemic injury comprises an acute phase and a chronic phase.

11. The method of claim 1, wherein the composition is administered 12 hours, to 48 hours after incidence of the ischemic injury.

12. The method of claim 1, further comprising administering the composition within 6 hours after the occurrence of the ischemic injury.

13. The method of claim 1, wherein the composition is administered one or more times daily.

14. The method of claim 1, wherein the composition is administered at least until one or more symptoms of the ischemic injury are resolved.

15. The method of claim 1, wherein the composition comprises a dose of 10-200 mg/kg of the Sigma receptor agonist.

16. The method of claim 1, wherein the composition is administered orally, subcutaneously, intraperitoneally, or intravenously.

17. The method of claim 1, wherein the composition comprises an effective amount of the Sigma receptor agonist to stimulate release of brain-derived neurotropic factor from neurons and/or to reduce one or more neurological deficits and/or symptoms.

18. An ischemic injury-reducing composition comprising an effective amount of a Sigma receptor agonist and one or more additional therapeutic agents and/or one or more pharmaceutically acceptable excipients to treat or reduce ischemic injury.

19. The composition of claim 18, wherein the one or more additional therapeutic agents comprise ketamine and/or selective serotonin reuptake inhibitors.

20. A method of producing a composition suitable for treating an ischemic injury in a subject, the method comprising: providing an effective amount of a Sigma receptor agonist capable of treating the ischemic injury; and combining the Sigma receptor agonist with one or more additional therapeutic agents and/or one or more pharmaceutically acceptable excipients to obtain the composition.
